(19) United States
(12) Patent Application Publication
(10) Pub. No.: US 2026/0144637 A1
(43) Pub. Date: May 28, 2026

(54) GUIDE CATHETER FOR AN IMPLANT DELIVERY APPARATUS

(71) Applicant: EDWARDS LIFESCIENCES CORPORATION, Irvine, CA (US)

(72) Inventors: Paolo Mario Tartara, Newport Beach, CA (US); Alyssa Joy Hamm (formerly Gross), Huntington Beach, CA (US); Corey Maurice Marshall, Newport Beach, CA (US); Kevin Gantz, Laguna Beach, CA (US); Yuanlong Du, Irvine, CA (US); Tri D. Tran, Fountain Valley, CA (US); Darshin S. Patel, Lake Forest, CA (US)

(73) Assignee: EDWARDS LIFESCIENCES CORPORATION, Irvine, CA (US)

(21) Appl. No.: 19/178,640

(22) Filed: Apr. 14, 2025

Related U.S. Application Data
(63) Continuation of application No. PCT/US2023/033745, filed on Sep. 26, 2023.
(60) Provisional application No. 63/380,796, filed on Oct. 25, 2022.

(51) Int. Cl. A61F 2/24; A61M 25/00; A61M 25/01; A61M 39/10

(52) U.S. Cl. CPC A61F 2/2439; A61M 25/0026; A61M 25/0147; A61M 39/10; A61M 2025/0004; A61M 2039/1072; A61M 2039/1077

(57) ABSTRACT

Devices and methods for coupling a retention member to a prosthetic implant are disclosed. As an example, a delivery apparatus can include a handle and a shaft extending distally from the handle. The shaft can have a longitudinal axis, a distal end, and a proximal end. The shaft can include a first inner surface defining a main lumen and a second inner surface defining a minor lumen. The delivery apparatus can further include a retention member partially disposed within the minor lumen. The retention member can include a retention element and an actuation element, wherein the retention member is selectively axially movable relative to the minor lumen and configured to releasably couple to a prosthetic implant at a target location.

### CROSS REFERENCE TO RELATED APPLICATION

This application is a continuation of PCT Patent Application No. PCT/US2023/033745 filed on Sep. 26, 2023, which claims the benefit of U.S. Provisional Patent Application No. 63/380,796, filed Oct. 25, 2022, each of which is incorporated by reference herein in its entirety.

### FIELD

The present disclosure relates to guide catheters for delivery apparatuses for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery or femoral vein) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

A guide catheter (which can also be referred to as a guide sheath) can be used for introducing a delivery apparatus, such as the prosthetic heart valve delivery apparatus described above, into the patient's vasculature. The guide catheter can include an elongated shaft that is inserted into the vasculature and a handle that remains outside the patient and can be used to manipulate the shaft. The delivery apparatus can be pushed through a main lumen of the guide catheter in order to help navigate the delivery apparatus to a target implantation site within the patient.

### SUMMARY

Described herein are prosthetic heart valves, docking devices, delivery apparatuses, and methods for implanting prosthetic heart valves. The disclosed prosthetic heart valves, docking devices, delivery apparatuses, and methods can, for example, provide stable positioning of a prosthetic implant during an implantation procedure, such that the positioning of the prosthetic implant can be adjusted and/or maintained during the implantation procedure. As such, the devices and methods disclosed herein can, among other things, overcome one or more of the deficiencies of typical prosthetic heart valves, docking devices and associated delivery apparatuses.

A delivery apparatus can comprise a handle and one or more shafts coupled to the handle.

In some examples, a delivery apparatus can comprise a shaft having a lumen exit that is angled, for example, to facilitate access to a patient's vasculature.

In some examples, a delivery apparatus can comprise one or more shafts having a main lumen, a minor lumen, and one or more pull wires disposed within the shaft(s), wherein the pull wire(s) are radially between the main lumen and the minor lumen.

In some examples, a delivery apparatus can comprise one or more shafts having a main lumen, a minor lumen, and pull wires disposed within the shaft(s), wherein the minor lumen is circumferentially between the pull wires.

In some examples, a delivery apparatus can comprise a shaft having an outer surface that includes a notch and/or a groove distal to a lumen exit.

In some examples, a delivery apparatus can comprise a retention member and one or more shafts having a lumen and an outer surface, wherein the retention member includes a first portion disposed within the lumen and a second portion disposed outside of the lumen and in contact with the outer surface.

In some examples, a delivery apparatus comprises a handle; a shaft extending distally from the handle and having a longitudinal axis, a distal end, and a proximal end, the shaft comprising a first inner surface defining a main lumen and a second inner surface defining a minor lumen; and a retention member partially disposed within the minor lumen, the retention member comprising a retention element and an actuation element, wherein the retention member is selectively axially movable relative to the minor lumen and configured to releasably couple to a prosthetic implant at a target location.

In some examples, a delivery apparatus comprises a handle; and a shaft extending distally from the handle and including a distal end and a proximal end, the shaft comprising a first inner surface defining a main lumen, a second inner surface defining a minor lumen, and a third inner surface defining a pull wire lumen, the shaft including a longitudinal axis that is coaxial with the main lumen; wherein the pull wire lumen is a first radial distance from the longitudinal axis, wherein the minor lumen is a second radial distance from the longitudinal axis, wherein the second radial distance is greater than the first radial distance.

In some examples, a delivery apparatus comprises a handle; and a shaft extending distally from the handle, the shaft having a longitudinal axis and multiple embedded lumens that are parallel to the longitudinal axis, the multiple embedded lumens including a main lumen, a minor lumen, and at least two pull wire lumens, wherein a center of the minor lumen is circumferentially between the at least two pull wire lumens.

In some examples, a delivery apparatus comprises a handle; a shaft having a distal end, a proximal end, and a longitudinal axis extending between the distal end and the proximal end, the shaft comprising: a first inner surface defining a main lumen, the main lumen coaxial with the longitudinal axis; a second inner surface defining a minor lumen; and a minor lumen exit coupled to the minor lumen, wherein the minor lumen exit comprises a distal surface that is angled relative to the longitudinal axis.

In some examples, a delivery apparatus comprises a handle; and a shaft extending distally from the handle and including an outer surface, a distal end, a proximal end and a longitudinal axis, the shaft comprising a first lumen that is coaxial with the longitudinal axis, a second lumen, and a positioning structure, the first lumen extending axially between the distal end and the proximal end, the second lumen having an exit that is axially offset from the distal end, the positioning structure disposed distal to the exit of the second lumen, the positioning structure comprising a notch.

In some examples, a delivery apparatus comprises a handle; a shaft extending distally from the handle and including an outer surface and a longitudinal axis, the shaft comprising a main lumen and a minor lumen; and a retention member partially disposed within the minor lumen, the retention member comprising a retention element and an actuation element, wherein the retention element is partially external to the minor lumen and contacts the outer surface of the shaft.

In some examples, a delivery apparatus comprises a handle; a shaft assembly extending distally from the handle and having a longitudinal axis, a distal end, and a proximal end, the shaft assembly comprising a first shaft defining a main lumen and a second shaft defining a minor lumen, wherein the first and second shafts are coupled together and are parallel to the longitudinal axis; and a retention member partially disposed within the minor lumen, the retention member comprising a retention element and an actuation element, wherein the retention member is selectively axially movable relative to the minor lumen and configured to releasably couple to a prosthetic implant at a target location.

In some examples, a delivery apparatus comprises one or more of the components recited in Examples 1-115, 127-137, 141, 159 below.

A shaft for a delivery apparatus can comprise multiple lumens.

In some examples, a shaft for a delivery apparatus, comprises a body having a longitudinal axis, the body comprising: a first inner surface defining a main lumen, the main lumen coaxial with the longitudinal axis; a second inner surface defining a minor lumen; and a third inner surface defining a pull wire lumen, wherein the pull wire lumen is a first radial distance from the longitudinal axis, wherein the minor lumen is a second radial distance from the longitudinal axis, wherein the second radial distance is greater than the first radial distance.

In some examples, a shaft for a delivery apparatus, comprises a body having multiple lumens that are embedded within the body, the multiple lumens comprising a main lumen, a minor lumen, and at least two pull wire lumens, wherein the multiple lumens are parallel, wherein a center of the minor lumen is circumferentially between the at least two pull wire lumens.

In some examples, a shaft for a delivery apparatus, comprises a body having a distal end, a proximal end, and a longitudinal axis extending between the distal end and the proximal end, the body comprising: a first inner surface defining a main lumen, the main lumen coaxial with the longitudinal axis; a second inner surface defining a minor lumen; and a minor lumen exit adjacent to the distal end of the shaft and coupled to the minor lumen, wherein the minor lumen exit comprises a distal surface that is angled relative to the longitudinal axis.

In some examples, a shaft for a delivery apparatus, comprises one or more of the components recited in Examples 138-140, 161 below.

An assembly can comprise an implant catheter and a delivery apparatus.

In some examples, an assembly can comprise an implant catheter including a prosthetic implant; and a guide catheter comprising: a shaft having a distal end and a proximal end, the shaft including a main lumen configured to receive a portion of the implant catheter therethrough and a minor lumen radially offset from the main lumen; and a retention member at least partially disposed within the minor lumen, the retention member configured to releasably couple to the prosthetic implant.

In some examples, an assembly comprises one or more of the components recited in Examples 116-126, 160 below.

A method can comprise coupling a retention member to a prosthetic implant.

In some examples, a method comprises inserting a guide catheter into a vessel of a patient; advancing a prosthetic implant coupled to a delivery apparatus through a main lumen of the guide catheter to an implant location; advancing a retention member through a minor lumen of the guide catheter; and coupling the retention member to the prosthetic implant.

In some examples, a method comprises inserting a shaft of a guide catheter into a vessel of a patient; translating a snare assembly at least partially disposed within a minor lumen of the shaft from a first position to a second position having a snare of the snare assembly distal to a distal end portion of the shaft; translating the snare assembly from the second position to a third position having the snare disposed around an outer surface of the distal end portion of the shaft; advancing a prosthetic implant coupled to a delivery apparatus through the snare and through a main lumen of the shaft to an implant location when the snare assembly is in the third position; and coupling the snare to the prosthetic implant.

In some examples, a method comprises inserting a shaft of a guide catheter having a snare of a snare assembly disposed around an outer surface of the shaft into a vessel of a patient, the snare assembly partially disposed within a minor lumen of the shaft; advancing a prosthetic implant coupled to a delivery apparatus through the snare and through a main lumen of the shaft to an implant location; and coupling the snare to the prosthetic implant.

In some examples, a method comprises inserting a guide catheter into a simulation; advancing a prosthetic implant coupled to a delivery apparatus through a main lumen of the guide catheter to a target location; advancing a retention member through a minor lumen of the guide catheter; and coupling the retention member to the prosthetic implant.

In some examples, a method comprises one or more of the components recited in Examples 142-158, 162 below.

The above method(s) can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a stage in an example mitral valve replacement procedure where a guide catheter and a guidewire are inserted into a blood vessel of a patient and navigated through the blood vessel and into a heart of the patient, towards a native mitral valve of the heart.

FIG. 2A schematically illustrates another stage in the example mitral valve replacement procedure where a docking device delivery apparatus extending through the guide catheter is implanting a docking device for a prosthetic heart valve at the native mitral valve.

FIG. 2B schematically illustrates another stage in the example mitral valve replacement procedure where the docking device of FIG. 2A is fully implanted at the native mitral valve of the patient and the docking device delivery apparatus has been removed from the patient.

FIG. 3A schematically illustrates another stage in the example mitral valve replacement procedure where a prosthetic heart valve delivery apparatus extending through the guide catheter is implanting a prosthetic heart valve in the implanted docking device at the native mitral valve.

FIG. 3B schematically illustrates another stage in the example mitral valve replacement procedure where the prosthetic heart valve is fully implanted within the docking device at the native mitral valve and the prosthetic heart valve delivery apparatus has been removed from the patient.

FIG. 4 schematically illustrates another stage in the example mitral valve replacement procedure where the guide catheter and the guidewire have been removed from the patient.

FIG. 5 is a side view of a guide catheter configured to receive a delivery apparatus and/or a tool and guide the delivery apparatus and/or the tool through a portion of a patient's vasculature, according to one example.

FIG. 6 is a side view of a shaft of the guide catheter of FIG. 5, with cross-sectional views of different portions of the shaft.

FIG. 7 is a side view of a distal end portion of the guide catheter of FIG. 5.

FIG. 8 is a side view of a distal end portion of a guide catheter, according to an example.

FIG. 9 is a side view of a delivery apparatus for a docking device, according to an example.

FIG. 10 is a perspective view of a docking device for use with the delivery apparatus of FIG. 9, according to an example.

FIG. 11 is a perspective view of a delivery apparatus for a prosthetic heart valve, according to an example.

FIG. 12 is a perspective view of a prosthetic heart valve for use with the delivery apparatus of FIG. 11, according to an example.

FIG. 13 schematically illustrates a stage in an example mitral valve replacement procedure using a retention member where the retention member is extended distally from a guide catheter positioned within a left atrium of a heart of a patient.

FIG. 14 schematically illustrates another stage in the example mitral valve replacement procedure using the retention member where the retention member is disposed around a shaft of the guide catheter.

FIG. 15 schematically illustrates another stage in the example mitral valve replacement procedure using the retention member where the retention member is disposed around the docking device in a relaxed state.

FIGS. 16A-16B schematically illustrate another stage in the example mitral valve replacement procedure using the retention member where the retention member is disposed around the docking device in a tensioned state.

FIGS. 17A-B schematically illustrates another stage in the example mitral valve replacement procedure using the retention member where a prosthetic heart valve delivery apparatus extending through the guide catheter is implanting a prosthetic heart valve in the implanted docking device at the native mitral valve and the retention member is disposed around the docking device in the tensioned state.

FIG. 18 schematically illustrates another stage in the example mitral valve replacement procedure using the retention member where the retention member is being removed from the docking device after the prosthetic heart valve in the implanted docking device at the native mitral valve.

FIG. 19 is a side view of a distal end portion of a delivery catheter, according to one example.

FIG. 20 is a perspective view of a distal end portion of a retention member, according to one example.

FIG. 21 is a perspective view of a distal end portion of a retention member, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

Described herein are examples of a steerable delivery apparatus (sometimes referred to as a steerable catheter) that can be used to navigate a subject's vasculature to deliver an implantable medical device (e.g., a prosthetic heart valve, a docking device), tools, agents, or other therapy to a location within the body of a subject. Examples of procedures in which the steerable catheters are useful include neurological, urological, gynecological, fertility (e.g., in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

In connection therewith, various systems, apparatuses, methods, or the like are described herein that, in some examples, can retain positioning of an implantable docking device at the location in the subject at various points throughout the procedure, including after the docking device is implanted as well as prior to and during implantation of a prosthetic heart valve within the docking device.

For example, a delivery apparatus (also referred to as “a guide catheter”) can be inserted into the subject and another delivery apparatus including a docking device retained therein can be navigated through a main lumen of the guide catheter toward a target location for the docking device. In some examples, the guide catheter can include a minor lumen for a retention member that can be releasably coupled to the docking device. The retention member can retain the docking device at a location within the patient (e.g., relative to the delivery apparatus, etc.) at various points throughout the procedure, including during implantation of the docking device, as well as prior to and/or during implantation of a prosthetic medical device (e.g., a prosthetic heart valve) within the docking device. The ability to adjust and/or maintain a positioning of the docking device throughout the procedure helps ensure that the docking device maintains its position relative to the native anatomy (e.g., prior to and/or during implantation of the prosthetic heart valve within the docking device).

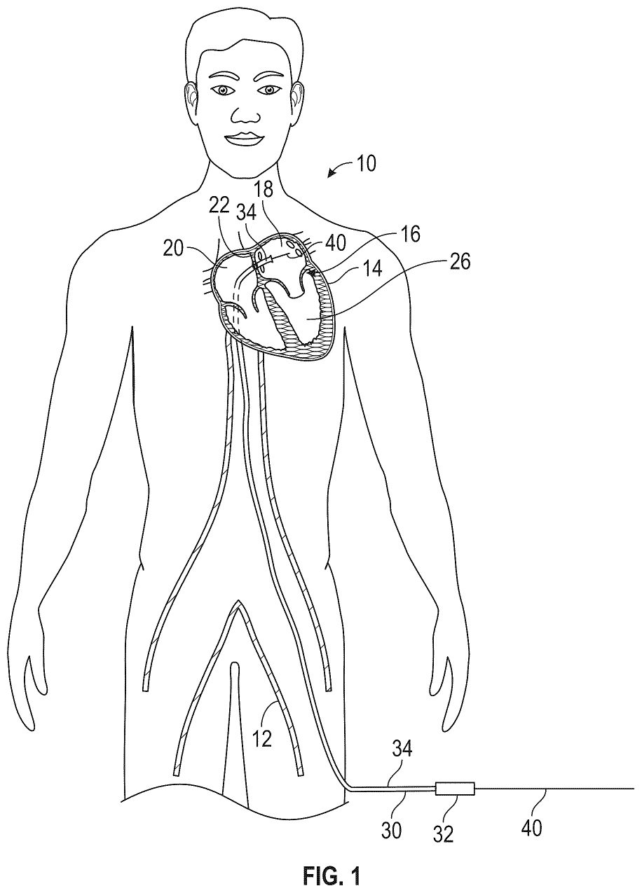

In some examples, the delivery apparatuses (or guide catheters) disclosed herein can be used to introduce one or more delivery apparatuses (or implant catheters) into the vasculature of a patient and guide the one or more delivery apparatuses at least partially through the vasculature toward a target implantation site. For example, FIGS. 1-4 schematically illustrate an example transcatheter heart valve replacement procedure which utilizes a guide catheter to guide a docking device delivery apparatus toward a native valve annulus and then a prosthetic heart valve delivery apparatus toward the native valve annulus. The docking device delivery apparatus is used to deliver a docking device to the native valve annulus and then the prosthetic heart valve delivery apparatus is used to deliver a transcatheter prosthetic heart valve inside the docking device.

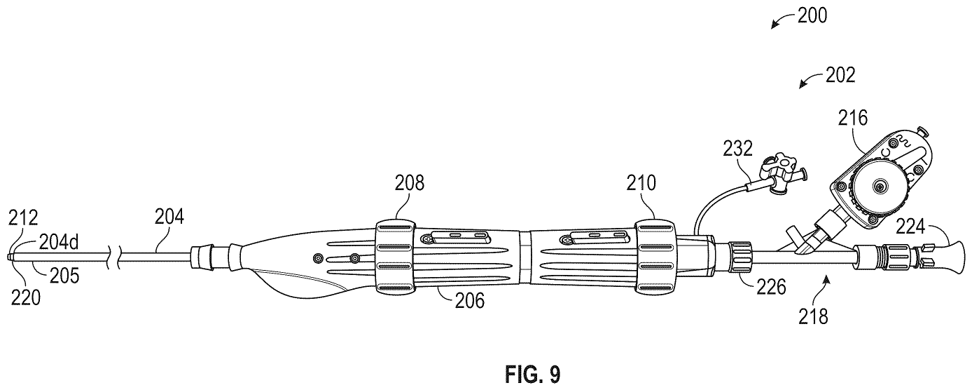

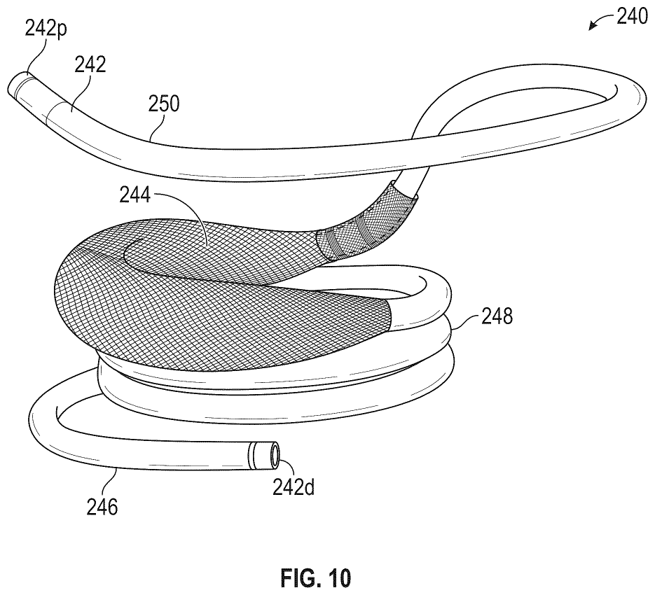

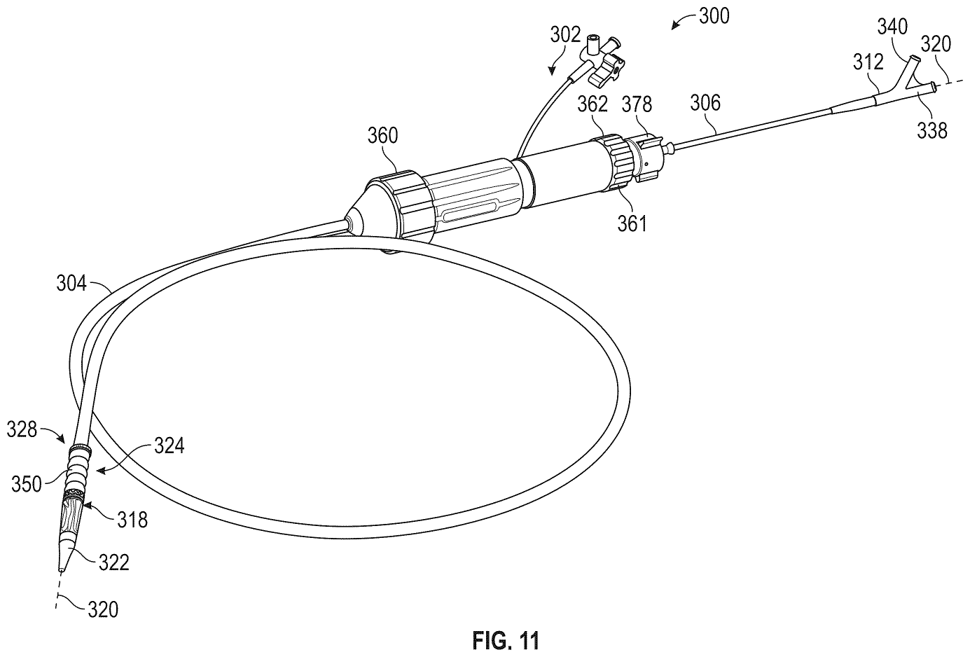

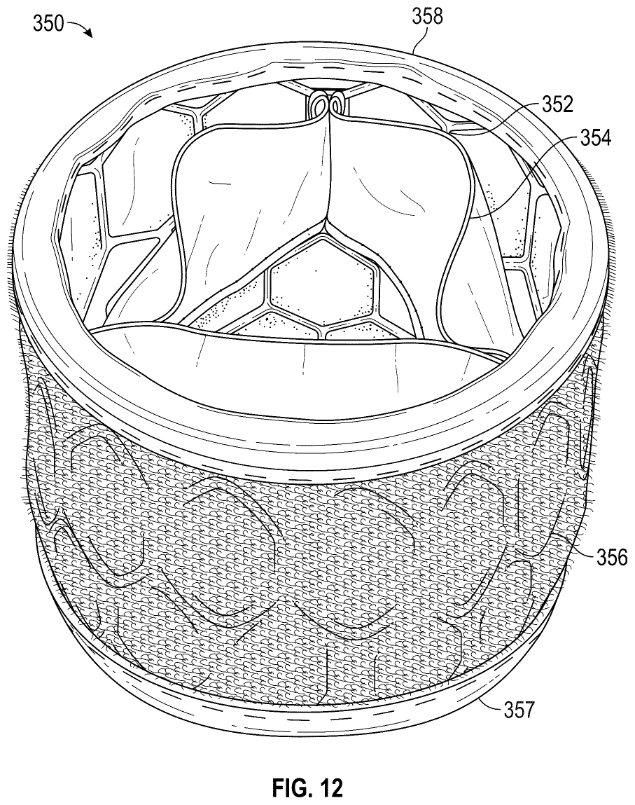

As introduced above, defective native heart valves may be replaced with transcatheter prosthetic heart valves. However, such prosthetic heart valves may not be able to sufficiently conform to the geometry of the native tissue (e.g., to the leaflets and/or annulus of the native heart valve) and may undesirably shift around relative to the native tissue, which can lead to paravalvular leakage. Thus, a docking device may be implanted first at the native valve annulus and then the prosthetic heart valve can be implanted within the docking device to help anchor the prosthetic heart valve to the native tissue and provide a seal between the native tissue and the prosthetic heart valve. An example delivery apparatus for delivering a docking device at a native heart valve is shown in FIG. 9, and an example docking device is shown in FIG. 10. An example delivery apparatus for delivering a prosthetic heart valve within a docking device at a native heart valve is shown in FIG. 11, and an example prosthetic heart valve is shown in FIG. 12.

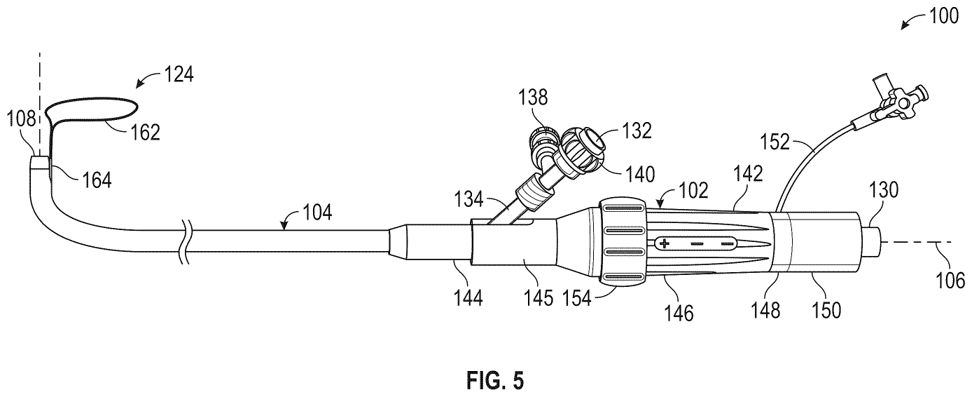

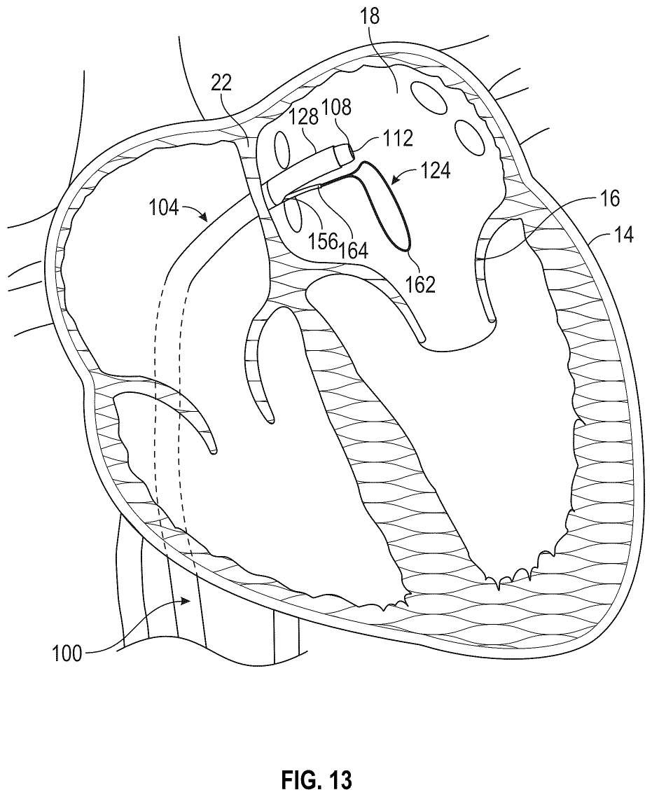

Example guide catheters are shown in more detail in FIGS. 5-8 and 19. In some examples, to prevent the docking device from undesirably shifting relative to the native tissue, the guide catheters include a retention member disposed within a minor lumen, as shown in FIGS. 5, 7, and 19. Additional examples of retention members are shown in FIGS. 20-21. Stages of the example transcatheter heart valve replacement procedure involving a retention member are shown in FIGS. 13-18.

### Examples of the Disclosed Technology

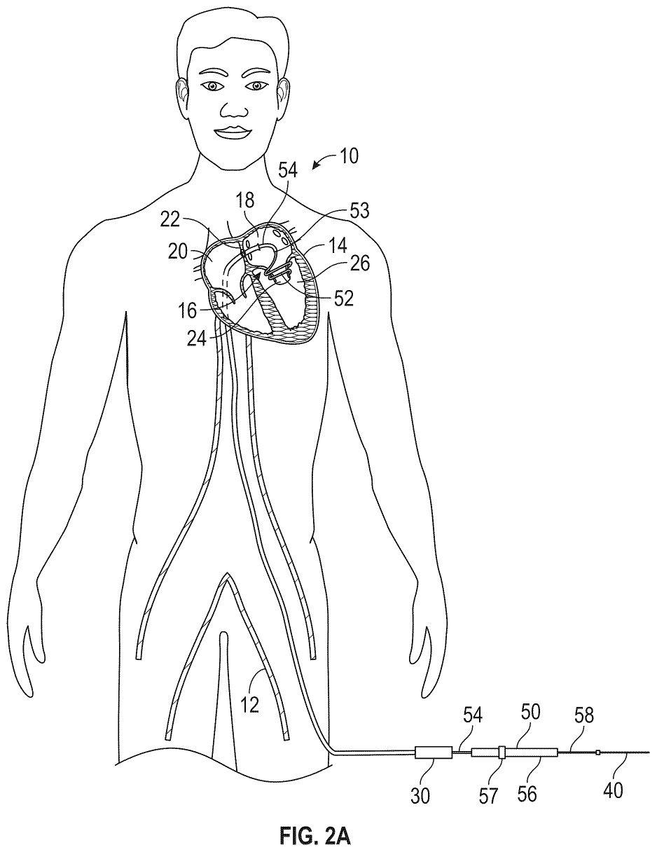

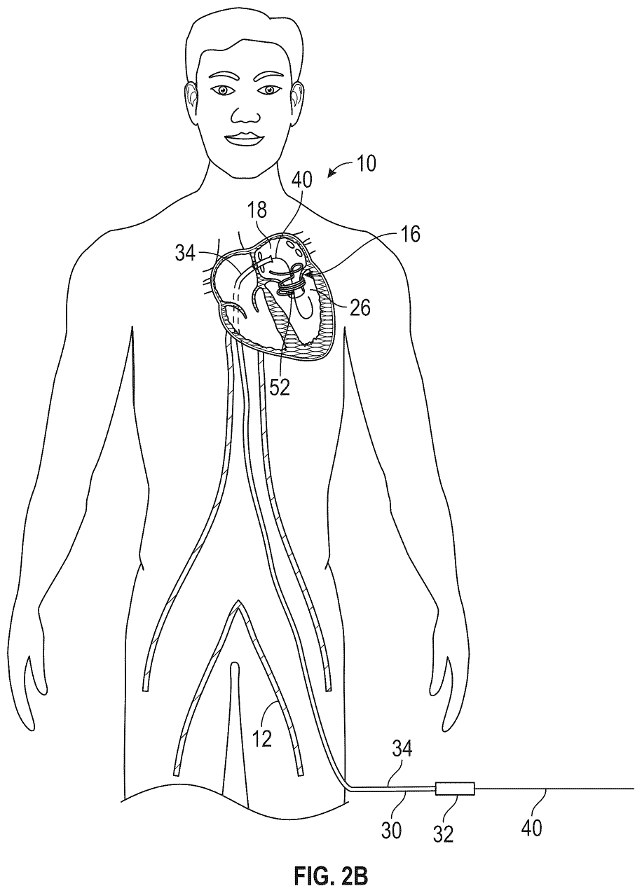

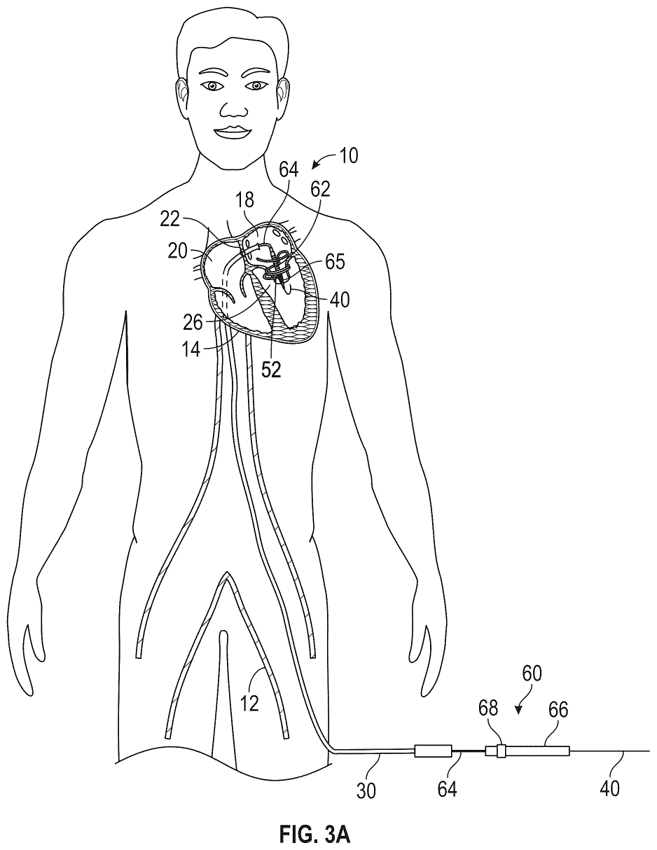

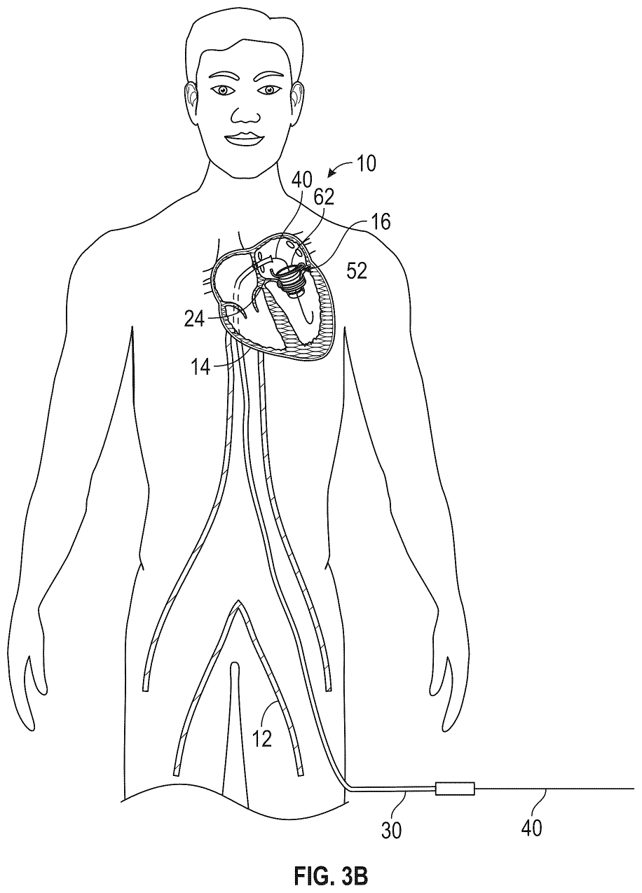

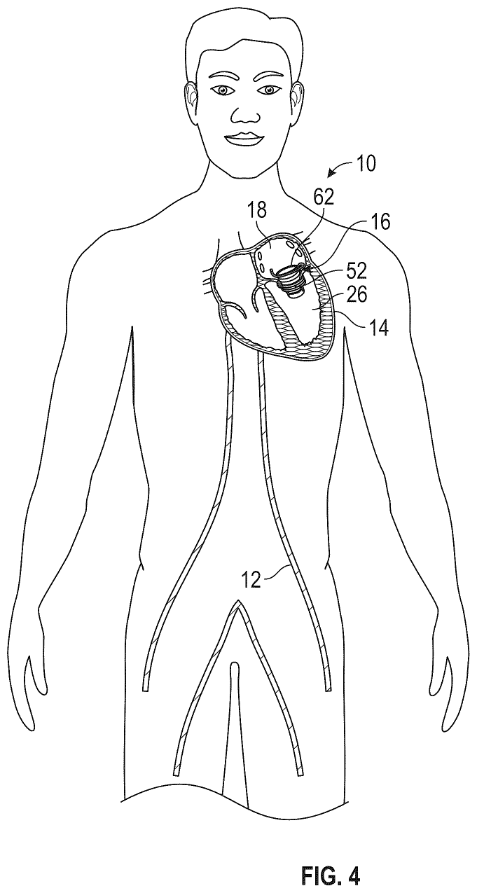

FIGS. 1-4 depict an example of a transcatheter heart valve replacement procedure (e.g., a mitral valve replacement procedure) which utilizes a docking device 52 and a prosthetic heart valve 62, according to one example. During the procedure, a user first creates a pathway to a patient's native heart valve using a guide catheter 30 (FIG. 1). The user then delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus). As shown, the heart 14 is illustrated schematically. For example, the anterior leaflet and chordae of the native mitral valve 16 are omitted for illustration purposes, such that only a portion of the posterior leaflet of the native mitral valve 16 is illustrated.

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (e.g., the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (e.g., the guide catheter 30, the docking device delivery apparatus 50, the prosthetic valve delivery apparatus 60, additional catheters, or the like) and their associated devices (e.g., docking device, prosthetic heart valve, and the like) to the implantation site within the heart 14, and thus may extend all the way through the blood vessel 12 and into a left atrium 18 of the heart 14 (FIG. 1) and in some examples, through the native mitral valve 16 and into a left ventricle of the heart 14.

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (e.g., through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts another stage in the example mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (e.g., native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (e.g., blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (e.g., the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO2020/247907, which is incorporated by reference herein in its entirety.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may then deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts another stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52.

FIG. 3B shows another stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts another stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

FIGS. 5-8 illustrate a guide catheter, which is referred to below as a guide catheter 100 (but can also be referred to herein as a “delivery apparatus” or an “introducer device”), according to one example. In some examples, the guide catheter 100 can be used as the guide catheter 30 in a prosthetic valve implantation procedure, as described above with reference to FIGS. 1-4. The guide catheter 100 can be configured to be inserted into a patient's vasculature and receive an implant catheter (and/or other delivery apparatus) therein in order to introduce the implant catheter into the patient's vasculature and at least partially guide the implant catheter to a target implantation site. Examples of implant catheters for prosthetic medical devices (referred to below as “delivery apparatus 200” and “delivery apparatus 300”) that can be received within the guide catheter 100 are shown in FIGS. 9 and 11 respectively, as described further below.

The guide catheter 100 can also be configured to receive a retention member (referred to below as “retention member 124,” “retention member 524” and “retention member 624”) therein such that the retention member can releasably couple to a prosthetic medical device at the target implantation site, for example, to retain the positioning of the prosthetic medical device relative to the implantation site and/or the guide catheter. Though the guide catheter 100 is described herein as being used with the delivery apparatus 200, the delivery apparatus 300, and the retention members 124, 524, 624, the guide catheter 100 can be configured to receive a variety of delivery apparatuses, implant catheters, or retention members, such as docking device delivery apparatuses, prosthetic heart valve delivery apparatuses, retention members, and/or delivery apparatuses for other medical devices or medical therapies.

The guide catheter 100 in the illustrated example comprises a handle 102, an elongated shaft 104 extending distally from the handle 102, and a longitudinal axis 106. In some examples, the shaft 104 can extend proximally into the handle 102.

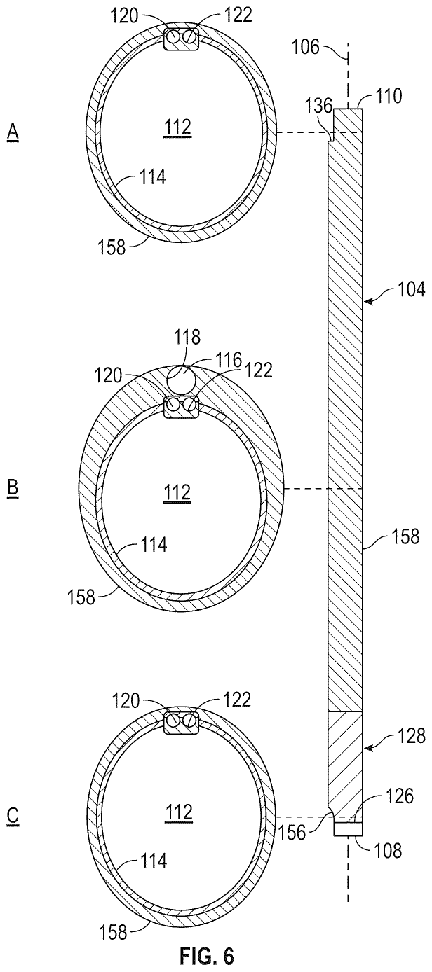

The shaft 104 can have multiple lumens extending along a length of the shaft 104, such that the lumens are parallel to the longitudinal axis 106. For example, the lumens can extend at least partially between a distal end 108 of the shaft 104 and a proximal end 110 of the shaft 104. In some examples, as shown in FIG. 6, the shaft 104 has a main (or primary) lumen 112 that is defined by a first inner surface 114 of the shaft 104. The shaft 104 has a minor (or secondary) lumen 116 that is defined by a second inner surface 118 of the shaft 104. The shaft 104 also has pull wire lumens 120 that are defined by third inner surfaces 122. In this way, the lumens 112, 116, and 120 are embedded within the shaft 104.

The main lumen 112 is configured to receive a delivery apparatus therein (such as any of the prosthetic device delivery apparatuses or implant catheters described herein). For example, the delivery apparatus can be configured to move (e.g., rotate within, translate axially, etc.) relative to the main lumen 112.

The minor lumen 116 is configured to receive a retention member therein (such as any of the retention members described herein). For example, the retention member can be configured to move (rotate within, translate axially, etc.) relative to the minor lumen 116. FIG. 5 illustrates a retention member 124 at least partially disposed within the minor lumen 116.

A pull wire (not shown) can extend through each of the pull wire lumens 120. In some examples, the pull wires can be coupled to a pull wire ring 126 disposed at or adjacent to the distal end 108 of the shaft 104. For example, adjusting a tension of the pull wires can adjust a curvature of a flex region 128 (FIG. 6) of the shaft 104. The flex region 128 can be disposed towards the distal end 108 of the shaft 104. In some instances, the shaft 104 (including its flex region 128) can be integrally formed as a single, unitary component. In some instances, as depicted in FIG. 6, the shaft 104 can comprise one or more segments (e.g., the flex region 128, other regions, etc.) that are formed as separate components that are coupled together (e.g., via fasteners, adhesive, mating features, and/or other means for coupling). In some examples, the flex region 128 can comprise a material that is more prone to flexing, bending, twisting, etc. than the remaining portion of shaft 104 (e.g., a polymer having relatively lower durometer hardness). This can enable the curvature of the flex region 128 to be adjusted or increased at a different rate than the remaining portion of the shaft 104 when the pull wires are tensioned. For example, the curvature of the flex region 128 can change at an increased rate relative to the proximal portion of the shaft 104 as the tension of the pull wires (not shown) is increased. The shaft 104 can also include one or more reinforcing braids or jackets that makes the shaft 104 more resistant to flexing, bending, twisting, etc., for example, to prevent one or more of the lumens from kinking or collapsing when the shaft 104 is manipulated.

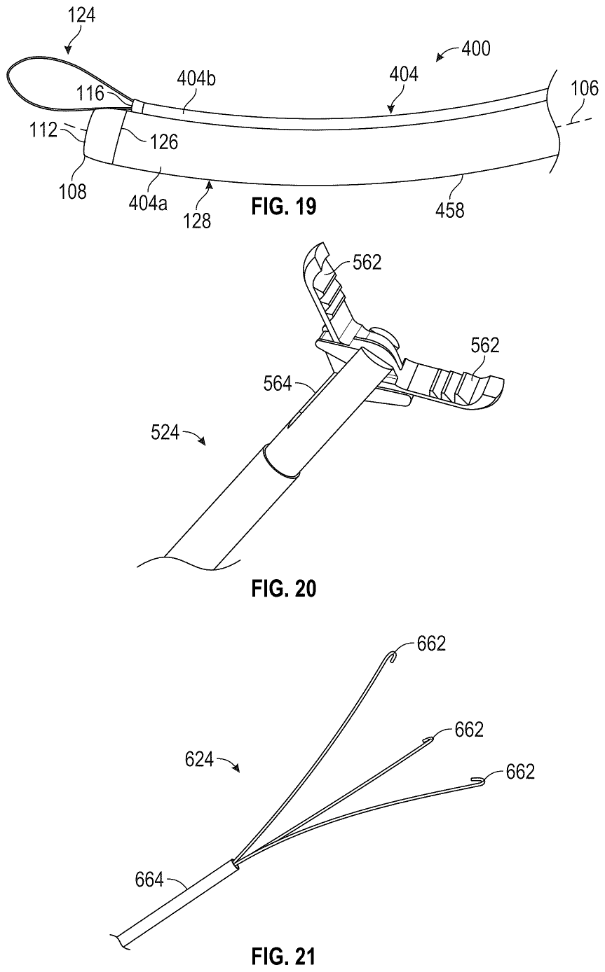

While guide catheter 100 is shown as having multiple lumens (main lumen 112, minor lumen 116, pull wire lumens 120) embedded within one shaft (shaft 104), it should be appreciated that each lumen, or a subset of these lumens, can be defined by inner surfaces or walls of multiple, discrete shafts that are coupled together in some examples. For example, FIG. 19 illustrates a distal end portion of a guide catheter 400 that comprises a shaft assembly 404 including a primary shaft 404a having the main lumen 112 and a secondary shaft 404b having the minor lumen 116. The secondary shaft 404b is coupled to the primary shaft 404a. The shaft assembly 404 can be coupled to a handle, such as handle 102.

The guide catheter 400 is substantially similar to guide catheter 100 described herein, although the guide catheter 400 includes the shaft assembly 404 comprising multiple shafts 404a, 404b with respective lumens 112, 116, instead of a single shaft 104 with multiple embedded lumens 112, 116. For example, an exit of the minor lumen 116 of both guide catheters 100, 400 is offset in an axial direction (e.g., parallel to longitudinal axis 106) from the distal end 108. For guide catheters having multiple shafts (e.g., guide catheter 400), the lumen(s) for pull wire(s) (not shown) can be included in the primary shaft 404a and/or the secondary shaft 404b. For example, when the pull wire(s) are included in the primary shaft 404a, the primary shaft 404a can include pull wire ring 126 and flex region 128, as shown in FIG. 19.

Referring again to FIGS. 5-8, the main lumen 112 can extend from the distal end 108 of the shaft 104 to a main inlet port 130 disposed at a proximal end of the handle 102. As depicted, the main inlet port 130 is coaxial and in fluid communication with the main lumen 112, such that a delivery apparatus can be inserted through the main inlet port 130 and into the main lumen 112. In some examples, the proximal end 110 of the shaft 104 is coupled to the main inlet port 130. Further, in some examples, the proximal end 110 of the shaft 104 can be spaced apart from the main inlet port 130 in the longitudinal direction, such that an inner surface of a wall of a portion of the handle 102 (e.g., at or adjacent the proximal end) can partially define the main lumen 112.

The minor lumen 116 can extend from the distal end 108 of the shaft 104 to a minor inlet port 132. In some examples, the minor inlet port 132 is distal to the main inlet port 130. For example, as shown in FIG. 5, the minor inlet port 132 can be disposed at or adjacent to a distal end of the handle 102. The minor inlet port 132 is coaxial and in fluid communication with the minor lumen 116, such that the retention member 124 (or any retention member described herein) can be inserted through the minor inlet port 132 and into the minor lumen 116.

In some examples, a connection shaft 134 is coupled to the minor inlet port 132 as well as an entrance 136 to the minor lumen 116 (or “minor lumen entrance 136”) disposed on the shaft 104. In this way, the connection shaft 134 (also referred to herein as the minor lumen 116 or an extension of the minor lumen 116) can partially define the minor lumen 116 proximal to the minor lumen entrance 136 of the shaft 104. As shown, the connection shaft 134 can be angled relative to the handle 102 to facilitate easier advancement of a retention member through the transition between the connection shaft 134 and the shaft 104. Further, in some examples, such as those with guide catheters having multiple, discrete shafts (an example of which depicted in FIG. 19), the connection shaft 134 can be omitted and the minor lumen entrance 136 can define the minor inlet port 132. For example, although not shown, the proximal end of the secondary shaft 404b of guide catheter 400 (FIG. 19) can define both a minor lumen entrance and a minor inlet port.

A flush port 138 can be fluidly coupled to minor lumen 116. For example, the flush port 138 can be coupled to the connection shaft 134 and include a lumen (not shown) that is in fluid communication with the extension of the minor lumen 116. In some examples, the flush port 138 can be fluidly coupled to the minor lumen 116 at other positions (e.g., along shaft 104, etc.). The flush port 138 can be configured to receive fluid through a lumen thereof. In this way, the flush port 138 can be used to maintain and/or improve the lubricity of the minor lumen 116, which can allow for improved movement of the retention member 124 relative to the minor lumen 116.

A lock 140 can be operatively coupled to the minor lumen 116 and can selectively limit and/or prevent movement of the retention member 124 relative to the minor lumen 116. For example, the lock 140 can be coupled to the connection shaft 134 distal and adjacent to the minor inlet port 132. In some instances, the lock 140 is also configured to prevent the backflow of fluid. For example, the lock 140 can be a tuohy borst adapter having a locking mechanism and a backflow prevention element or seal.

In some examples, the handle 102 can also include an outer housing 142. In some instances, the housing 142 can be integrally formed as a single, unitary component. In other instances, as depicted, the housing 142 can comprise one or more segments that are formed as separate components that are coupled together (e.g., via fasteners, adhesive, mating features, and/or other means for coupling). For example, the housing 142 can comprise a distal nosecone portion 144, a proximal nosecone portion 145, a main body portion 146, an adaptor portion 148, and a seal housing portion 150.

The shaft 104 can extend into the handle 102 through at least the distal nosecone portion 144 and the proximal nosecone portion 145. The minor inlet port 132 can be coupled to the proximal nosecone portion 145 of the housing 142. In this example, the minor inlet port 132 of the minor lumen 116 is axially between the distal nosecone portion 144 and the main body portion 146. However, in some examples, the minor inlet port 132 can be coupled to other portions of the housing 142.

Within the housing 142, the handle 102 can include one or more seals and a steering mechanism (including the pull wires) (not shown). For example, the seal housing portion 150 (which can also be referred to as a “seal stack”) comprises one or more seals contained therein. As depicted, the seal housing portion 150 is disposed distal and adjacent to the main inlet port 130. The seal(s) disposed within the seal housing portion 150 can be configured to fluidly seal the main lumen 112 of the guide catheter 100 from the external environment. For example, the one or more seals of the seal housing portion 150 can be configured to prevent blood from a patient in which the guide catheter 100 is inserted from exiting the guide catheter 100 and prevent air from the environment from entering the guide catheter 100 (e.g., through the inlet port 130). The seal(s) can include a variety of types of seals, such as a duckbill seal, a flapper seal, an umbrella valve, a cross-slit valve, a dome valve, or the like.

The adaptor portion 148 is disposed distal and adjacent to the seal housing portion 150 and proximal and adjacent to the main body portion 146. A flush port 152 can be coupled to the housing 142, for example, at the adaptor portion 148. The flush port 152 can be fluidly coupled to main lumen 112, for example, through a lumen (not shown) of the adaptor portion 148 that is in fluid communication with the main lumen 112. In some examples, the flush port 152 can be fluidly coupled to the main lumen 112 at other positions (e.g., coupled to other portions of the housing 142, etc.). The flush port 152 can be configured to receive fluid through a lumen thereof. In this way, the flush port 152 can be used to maintain and/or improve the lubricity of the main lumen 112, which can allow for improved movement of a delivery apparatus (such as those described herein) relative to the main lumen 112.

As introduced above, the handle 102 can include a steering mechanism configured to adjust the curvature of the flex region 128 of the shaft 104 (as such, the shaft 104 can be referred to as a steerable shaft). In the illustrated example, the main body portion 146 is disposed adjacent and distal to the adaptor portion 148 and can include an adjustment member, such as the illustrated rotatable knob 154. The main body portion 146 can house internal flex mechanisms of the guide catheter 100 which are operatively coupled to the rotatable knob 154. In some examples, the flex mechanisms, and thus the knob 154, can be operatively coupled to the proximal end portion of one or more pull wires. The pull wires can extend distally from the handle 102 through the pull wire lumens 120 of the shaft 104 and have a distal end portion affixed to the pull wire ring 126 disposed at or near the distal end 108 of the shaft 104. Rotating the knob 154 can increase or decrease the tension in the pull wires, thereby adjusting the curvature of the flex region 128 of the shaft 104. Further details on steering or flex mechanisms for a delivery apparatus can be found in U.S. Pat. No. 9,339,384, which is incorporated by reference herein in its entirety.

FIG. 6 illustrates multiple cross-sectional views of the shaft 104. Specifically, FIG. 6 includes view A taken through a proximal end portion of the shaft 104, view B taken through a central portion of the shaft 104, and view C taken through a distal end portion of the shaft 104. View B includes both the main lumen 112 and the minor lumen 116, whereas views A and C do not include the minor lumen 116. Specifically, view A is proximal to the minor lumen entrance 136 and view C is distal to an exit 156 of the minor lumen 116 (or “minor lumen exit 156”). As shown in the cross-sectional views of FIG. 6, the surfaces 114, 118, 122 generally define continuous round shapes, such as oval, circular or the like, such that the lumens 112, 116, 120 can each be a round shape.

As depicted in FIG. 6, the main lumen 112 is coaxial with the longitudinal axis 106. In some examples, as shown, the minor lumen 116 is offset from the longitudinal axis 106 (and the main lumen 112) in the radial direction. In particular, the minor lumen 116 can be radially outward of the main lumen 112. In some examples, as depicted, the minor lumen 116 is also radially outward of the pull wire lumens 120. In the illustrated example, at least a portion of the shaft 104 is disposed between lumens in at least the radial direction, such that each lumen is separate from the other lumens. For example, as depicted, the surfaces 114, 118, 122 defining the lumens 112, 116, 120 are separate and distinct.

Further, in some examples, the center of the minor lumen 116 can be circumferentially between the pull wire lumens 120, as depicted. In some instances, positioning the center of the minor lumen 116 circumferentially between the pull wire lumens 120, rather than offset from the pull wire lumens 120 in the circumferential direction, can decrease or minimize the impact the minor lumen 116 may otherwise have on the steerability of the shaft 104. For example, offsetting the minor lumen 116 in a circumferential direction (instead of the positioning shown in FIG. 6) may skew the curvature of the flex region 128 of the shaft 104 and/or require an increased amount of force to maintain the curvature of the flex region 128. In some examples, the minor lumen 116 can be positioned in a circumferentially offset position to impact the steerability of the shaft 104. For example, the minor lumen 116 can be offset from a center of the pull wire lumen(s) 120 in the circumferential direction by 90 degrees, 180 degrees, etc.

In some examples, although not shown, any shaft or shaft assembly described herein can include more than two pull wire lumens 120 (e.g., three pull wire lumens, etc.). In these examples, the center of the minor lumen 116 can be circumferentially between at least two of the pull wire lumens 120. For instance, in examples including three pull wire lumens, the center of the minor lumen can either be circumferentially aligned with one of the pull wire lumens (e.g., a circumferentially central pull wire lumen) or offset from the pull wire lumens, as long as the center of the minor lumen 116 is still circumferentially between at least two of the pull wire lumens.

The shaft 104 includes an outer surface 158. As shown in the cross-sectional views of FIG. 6, the outer surface 158 is generally defines a continuous round shape, such as oval, circular or the like. Additionally, the outer surface 158 generally maintains the same shape along its length, although the outer surface 158 of the shaft 104 has a larger shape at a location where the minor lumen 116 is disposed (for example, a central portion of the shaft 104 shown in view B as compared to views A and C). This is distinct from an outer surface 458 of the shaft assembly 404 of guide catheter 400 (FIG. 19), which generally defines a discontinuous round shape, such as adjacent or adjoining oval or circular shapes (e.g., having an intersection point at the location where the primary and secondary shafts 404a, 404b are coupled together).

As introduced above, the shaft 104 can include one or more reinforcing braids, coils, or jackets disposed within the shaft 104. For example, the main shaft 112 can include a reinforcing braid or jacket (not shown) disposed within the shaft 104 and around the main shaft 112. The reinforcing jacket can extend along a length of the shaft 104, including within and/or proximal to the flex region 128. Additionally and/or alternatively, in some examples, one or more of the inner surfaces 114, 118, 122 of the lumens 112, 116, 120 can include a reinforcing liner (e.g., a braid, a coil, etc.) to prevent the lumen from collapsing or kinking when the curvature of the shaft 104 is adjusted. For example, the shaft 104 can include a coil of material (e.g., a flat or rounded material) that can circumferentially surround or define the lumen and/or at least partially extend along a length of the lumen.

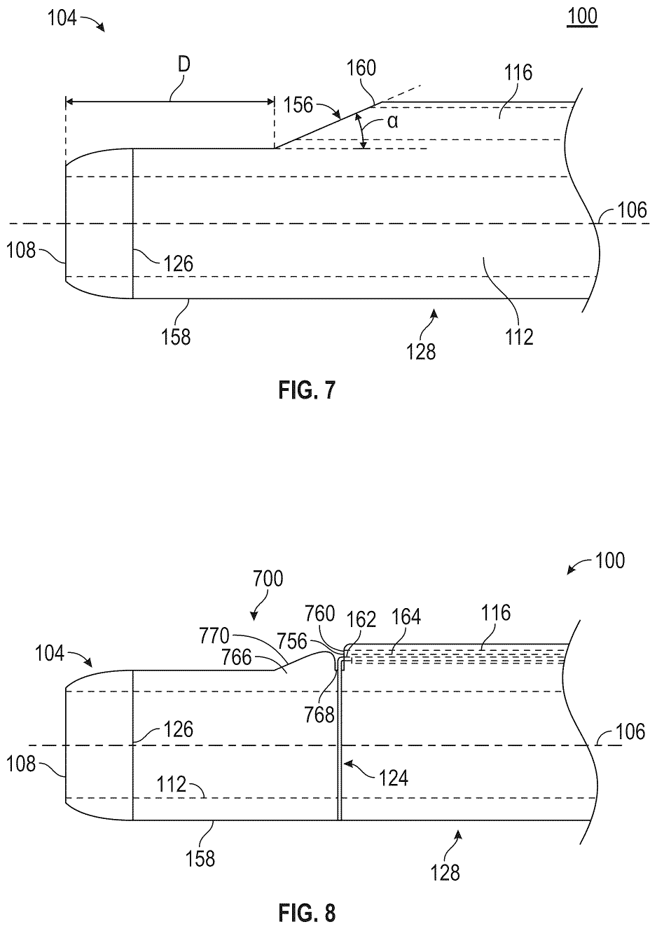

In the illustrated example, the minor lumen exit 156 is proximal to the distal end 108 of the shaft 104 (and an exit of the main lumen 112). As shown in FIG. 7, the minor lumen exit 156 is offset from the distal end 108 of the shaft 104 by a distance D. Specifically, distance D is the distance between the distal end 108 of the shaft 104 and a distal-most portion of the minor lumen exit 156. As explained further below, this spacing enables proper positioning of the shaft 104 and the retention member 124 (broadly, guide catheter 100) during an implantation procedure (such as the one described above in FIGS. 1-4). With this spacing, the minor lumen exit 156 can be disposed within the flex region 128 of the shaft 104 and, in some examples, distal to a reinforcing braid surrounding the main lumen 112. Specifically, in these examples, the minor lumen exit 156, which is disposed within the flex region 128, can be proximal to the pull wire ring 126 and distal to the reinforcing jacket. By positioning the minor lumen exit 156 distal to the jacket, the minor lumen 116 can be offset from the distal end 108 of the shaft 104 without the minor lumen 116 piercing or extending through the jacket, for example, in the radial direction.

In some examples, as shown in FIG. 7, the minor lumen exit 156 is tapered and includes a distal surface 160 that is angled relative to the longitudinal axis 106 (and in some instances, relative to the outer surface 158 of the shaft 104). Specifically, the distal surface 160 is disposed at an angle α. The angle α can be less than 90 degrees, and in some examples, about 20 degrees. As such, a radial size of the minor lumen exit 156 (therefore, the shaft 104) gradually increases in the proximal direction. This can allow the guide catheter 100 to be inserted through various tissue, for example, through a wall of a heart, without the minor lumen exit 156 interfering with, snagging, or getting caught on the tissue. For example, when the guide catheter 100 is advanced through the atrial septum 22 of a patient, the angled surface 160 of the minor lumen exit 156 allows the radial size of the shaft 104 (e.g., at the atrial septum 22) to gradually increase as the shaft 104 is advanced distally through the atrial septum 22.

FIGS. 7 and 8 illustrate the main lumen 112 and the minor lumen 116 in dashed lines. The pull wire lumens 120 (and pull wires disposed therein) are omitted for purposes of illustration. As introduced above, the minor lumen 116 is configured to receive a retention member 124 therein. The retention member 124 can be configured to selectively and/or releasably couple to an implantable prosthetic medical device (or prosthetic implant) at a target location, for example, during an implantation procedure. Specifically, the retention member 124 can be configured to reduce movement and/or retain a position of the prosthetic implant relative to the target location, as explained in more detail below.

As shown in FIGS. 5 and 8, the retention member 124 comprises a retention element 162 and an actuation element 164. The retention element 162 is the portion of the retention member 124 that engages with the prosthetic implant and the actuation element 164 can actuate the retention element 162.

In some examples, as depicted, the retention element 162 comprises a loop (or snare) of wire, suture, etc. and the actuation element 164 comprises an outer shaft that is slidable relative to the retention element 162. For example, sliding the outer shaft 164 relative to the loop 162 can decrease the size of the loop 162, such that the loop 162 can retain or constrain a portion of the prosthetic device therein. As shown in FIGS. 5 and 8, the retention member 124 comprises a retention element 162 having a single loop having a 90-degree bend and a slidable outer shaft 164. In some instances, the retention element 162 comprises a loop or snare that does not include such a bend (e.g., the retention element 162 is in) or includes bends at other angles. Further, in some instances, the retention element 162 can comprise multiple loops.

In addition to retention member 124, various types of retention members can be used with and/or be included in the guide catheters described herein (such as guide catheter 100, guide catheter 400, etc.). In some examples, as shown in FIGS. 20-21, retention members can comprise different types of grasping devices or forceps. For example, the retention member 524 of FIG. 20 includes a retention element 562 that comprises gripping claws, talons, or teeth that can be actuated by actuation element 564. As shown, actuation element 564 comprises a pull shaft or wire, that when pulled, draws the retention element 562 closed around a target object, such as the prosthetic implant. The retention member 624 of FIG. 21 includes a retention element 662 that comprises multiple barbed prongs that can be actuated by actuation element 664. As shown, actuation element 664 comprises an outer shaft (similar to actuation element 164) that is slidable relative to the retention element 662. For example, sliding the outer shaft 664 relative to the barbed prongs 662 can draw the prongs 662 together around the target object, such as the prosthetic implant, in order for the barbs of the prongs 662 grasp, retain, and/or constrain the object.

In some examples, a retention member (such as retention member 124 or any retention member described herein) can be disposed in an insertion configuration during insertion of a guide catheter (such as guide catheter 100 or any guide catheter described herein) into the body of a patient. The insertion configuration enables the guide catheter 100, for example, to be inserted such that the retention member 124, for example, does not interfere with, snag, or otherwise catch on tissue as the guide catheter 100 is advanced towards the target location. In some instances, the insertion configuration is a fully separate or external configuration and includes the retention member 124 being fully external to the guide catheter 100 (and the minor lumen 116). When the external insertion configuration is used, the retention member 124 is inserted into the minor lumen 116 after the guide catheter 100 is inserted into a body and advanced to the target location.

In some instances, an insertion configuration of a retention member can be a fully internal configuration. In this configuration, the retention member 124, for example, can be fully disposed within the minor lumen 116 during insertion. For example, the retention member 124 can be fully disposed within the minor lumen 116 and a distal end of the retention member 124 can be proximal to the minor lumen exit 156. After the guide catheter 100 (including the retention member 124 disposed within the minor lumen 116) is advanced to or adjacent to the target location, the retention member 124 can be advanced distally relative to the guide catheter 100.

In some examples, an insertion configuration of a retention member can be a partially internal configuration. In this configuration, the retention member 124, for example, can be partially disposed within the minor lumen 116 during insertion, such that at least a distal end of the retention member 124 (e.g., the retention element 162) is distal to the minor lumen exit 156.

In some examples of the partially internal insertion configuration, the retention element 162 can be disposed around the outer surface 158 of the shaft 104, for example, at a location distal to the minor lumen exit 156. In this manner, the retention element 162 can surround the main lumen 112 in a circumferential direction. Additionally, the actuation element 164 can be actuated, such that the retention element 162 is tightly engaged with the outer surface 158. Specifically, the diameter of the loop 162 can be decreased by advancing the outer shaft 164 distally relative to the loop 162, such that the loop 162 is secured against the outer surface 158 to limit movement of the loop 162 relative to the shaft 104, for example, during insertion of the guide catheter 100.

In some examples, guide catheters (such as any guide catheter described herein) can optionally include a positioning structure, such as a notch, adjacent the minor lumen exit for positioning the retention member in a fixed position relative to the outer surface of the shaft in the partially internal insertion configuration. For example, FIG. 8 illustrates the guide catheter 100 as having a positioning structure 700 for housing a retention member (e.g., retention member 124) in a partially internal insertion configuration. Any guide catheter described herein having a minor lumen can include a positioning structure 700.

FIG. 8 also illustrates the guide catheter 100 as having a “flat” minor lumen exit 756. Specifically, rather than being angled at an angle of less than 90 degrees (such as minor lumen exit 156), the minor lumen exit 756 includes a distal surface 760 that is parallel to the exit of the main lumen 112. In other words, the distal surface 760 is angled relative to the longitudinal axis 106 (and in some instances, relative to the outer surface 158 of the shaft 104) at an angle of 90 degrees. It should be appreciated that the positioning structure 700 can be included on guide catheters having an angled minor lumen exit, such as minor lumen exit 156. Additionally, guide catheters described herein can include a flat minor lumen exit without including a positioning structure (FIG. 19).

The positioning structure 700 can include a projection 766 and a notch 768. The projection 766 can extend radially from the outer surface 158 of the shaft 104. As shown in FIG. 8, the projection 766 is distal to the terminal portion or exit of the minor lumen 116. The notch 768 is disposed axially between the projection 766 and the minor lumen exit 756 and is configured to retain a retention element of a retention member therein. In some instances, as depicted, the surface of the notch 768 and the outer surface 158 (at a location distal to the projection 766) are the same radial distance from the longitudinal axis 106. In some instances, although not shown, the surface of the notch 768 can be recessed in the radial direction. That is, the surface of the notch 768 can have a shorter radial distance from the longitudinal axis 106 than the outer surface 158 (e.g., at a location distal to the projection 766). In these instances, the notch 768 can be referred to as a grooved notch in the outer surface 158.

The projection 766 includes a distal surface 770 that is angled relative to the longitudinal axis 106 (and in some instances, relative to the outer surface 158 of the shaft 104). Specifically, the distal surface 760 can be disposed at an angle of less than 90 degrees, and in some examples, about 20 degrees. As such, a radial size of the projection 766 (therefore, the shaft 104) gradually increases in the proximal direction. This can allow the guide catheter 100 to be inserted through various tissue, for example, through a wall of a heart, without the positioning structure 700, the minor lumen exit (e.g., minor lumen exit 756), and the retention member (e.g., retention member 124) from interfering with, snagging, or getting caught on the tissue. For example, when the guide catheter 100 is advanced through the atrial septum 22 of a patient, the angled surface 770 of the positioning structure 700 allows the radial size of the shaft 104 (e.g., at the atrial septum 22) to gradually increase as the shaft 104 is advanced distally through the atrial septum 22.

As shown in FIG. 8, the projection 766 is approximately as wide as the minor lumen exit 756 in the circumferential direction. In these examples, the projection 766 can be aligned circumferentially with the minor lumen exit 756. In some examples, the projection 766 can extend circumferentially around the outer surface 158 of the shaft 104.

FIG. 9 illustrates a delivery apparatus 200 configured to implant a docking device, such as docking device 240 (FIG. 10) described below or other docking devices, to a target implantation site in a patient, according to one example. For example, the delivery apparatus 200 can be used as the docking device delivery apparatus 50 in a prosthetic valve implantation procedure, as described above with reference to FIG. 2A. The delivery apparatus 200 can also be referred to as a “dock delivery catheter” or “dock delivery system.”

As shown, the delivery apparatus 200 can include a handle assembly 202 and a delivery sheath 204 (also referred to as the “delivery shaft” or “outer shaft” or “outer sheath”) extending distally from the handle assembly 202. The handle assembly 202 can include a handle 206 including one or more knobs, buttons, wheels, and/or other means for controlling and/or actuating one or more components of the delivery apparatus 200. For example, in some examples, as shown in FIG. 7, the handle 206 can include knobs 208 and 210 which can be configured to steer or control flexing of the delivery apparatus 200 such as the delivery sheath 204 and/or the sleeve shaft 220 described below.

In certain examples, the delivery apparatus 200 can also include a pusher shaft 212 and a sleeve shaft 220, both of which can extend through an inner lumen of the delivery sheath 204 and have respective proximal end portions extending into the handle assembly 202.

As described below, a distal end portion (also referred to as “distal section”) of the sleeve shaft 220 can be configured to cover (e.g., surround) the docking device 240 (see FIG. 8). For example, the docking device 240 can be retained inside the sleeve shaft 220, which is further retained by a distal end portion 205 of the delivery sheath 204, when navigating through a patient's vasculature.

Additionally, the distal end portion 205 of the delivery sheath 204 can be configured to be steerable. In one example, by rotating a knob (e.g., 208 or 210) on the handle 206, a curvature of the distal end portion 205 can be adjusted so that the distal end portion 205 of the delivery sheath 204 can be oriented in a desired angle. For example, to implant the docking device 240 at the native mitral valve location, the distal end portion 205 of the delivery sheath 204 can be steered in the left atrium so that at least a portion of the sleeve shaft 220 and the docking device 240 retained therein can extend through the native mitral valve annulus at a location adjacent the posteromedial commissure.

In certain examples, the pusher shaft 212 and the sleeve shaft 220 can be coaxial with one another, at least within the delivery sheath 204. In addition, the delivery sheath 204 can be configured to be axially movable relative to the sleeve shaft 220 and the pusher shaft 212. As described further below, a distal end of the pusher shaft 212 can be inserted into a lumen of the sleeve shaft 220 and press against the proximal end of the docking device 240 retained inside the sleeve shaft 220.

After reaching a target implantation site, the docking device 240 can be deployed from the delivery sheath 204 by manipulating the pusher shaft 212 and sleeve shaft 220 using a hub assembly 218, as described further below. For example, by pushing the pusher shaft 212 in the distal direction while holding the delivery sheath 204 in place or retracting the delivery sheath 204 in the proximal direction while holding the pusher shaft 212 in place, or pushing the pusher shaft 212 in the distal direction while simultaneously retracting the delivery sheath 204 in the proximal direction, the docking device 240 can be pushed out of a distal end 204d of the delivery sheath 204, thus changing from a delivery configuration to a deployed configuration (see FIG. 8). In certain examples, the pusher shaft 212 and the sleeve shaft 220 can be actuated independently of each other.

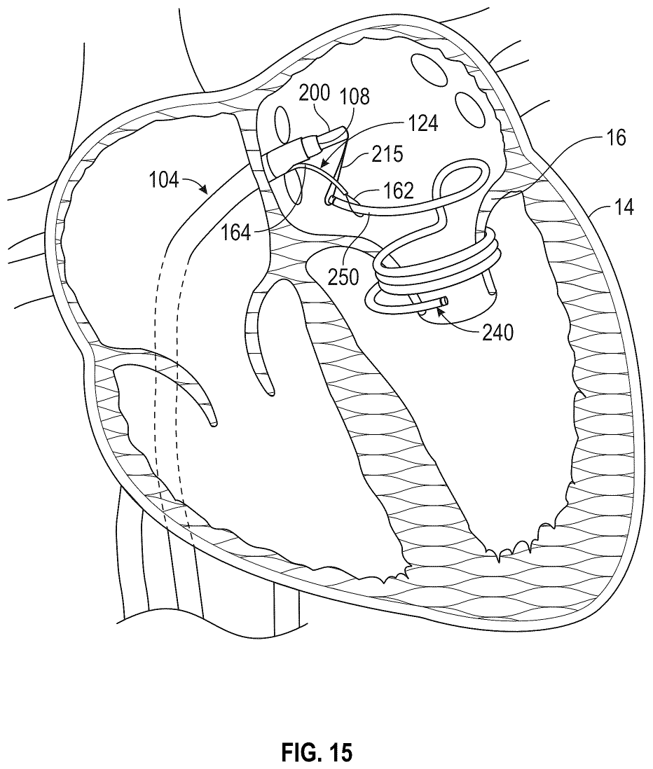

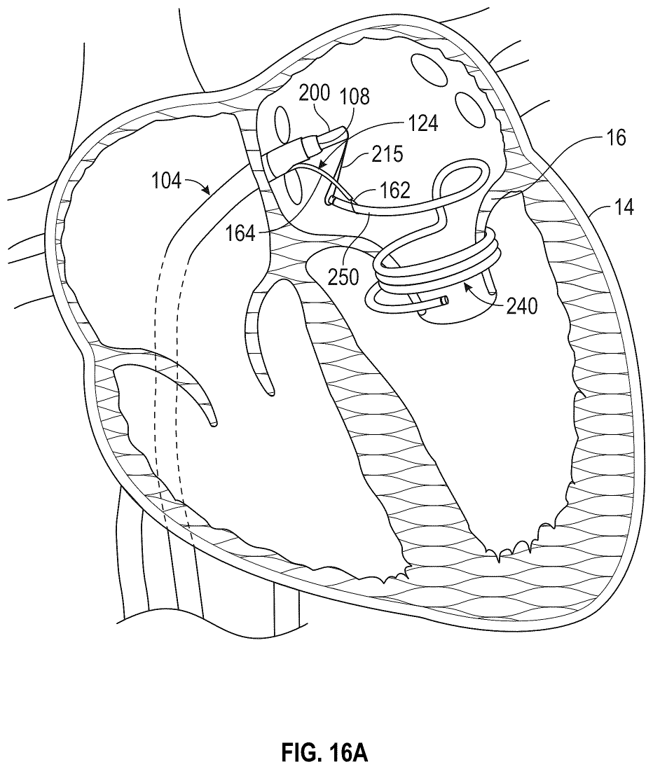

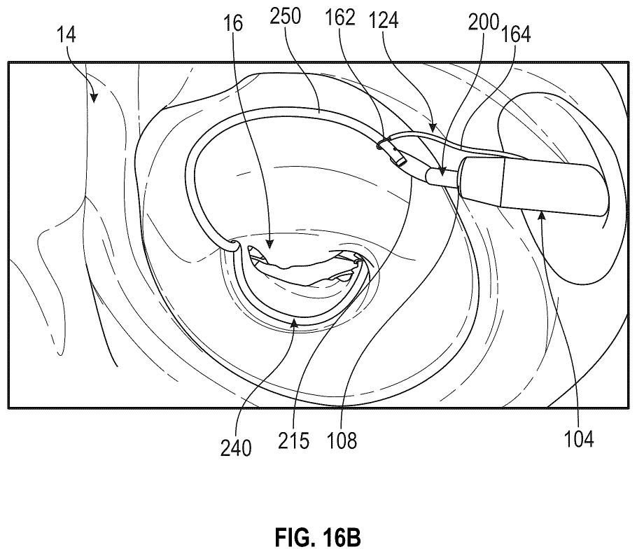

During delivery, the docking device 240 can be coupled to the delivery apparatus 200 via a release suture 215 (FIGS. 15-16B) (or other retrieval line comprising a string, yarn, or other material that can be configured to be tied around the docking device 240 and cut for removal) that extends through the pusher shaft 212. In one specific example, the release suture 215 can extend through the delivery apparatus 200, e.g., through an inner lumen of the pusher shaft 212, to a suture lock assembly 216 of the delivery apparatus 200.

The handle assembly 202 can further include a hub assembly 218 to which the suture lock assembly 216 and a sleeve handle 224 are attached. The hub assembly 218 can be configured to independently control the pusher shaft 212 and the sleeve shaft 220 while the sleeve handle 224 can control an axial position of the sleeve shaft 220 relative to the pusher shaft 212. In this way, operation of the various components of the handle assembly 202 can actuate and control operation of the components arranged within the delivery sheath 204. In some examples, the hub assembly 218 can be coupled to the handle 206 via a connector 226.

The handle assembly 202 can further include one or more flush ports (e.g., flush port 232 is shown in FIG. 7) to supply flush fluid to one or more lumens arranged within the delivery apparatus 200 (e.g., annular lumens arranged between coaxial components of the delivery apparatus 200).

Further details on delivery apparatus/catheters/systems (including various examples of the handle assembly) that are configured to deliver a docking device to a target implantation site can be found in International Application No. PCT/US2020/036577 and in U.S. Patent Publication Nos. 2018/0318079 and 2018/0263764, which are all incorporated by reference herein in their entireties.

FIG. 10 illustrates a docking device 240, according to one example. The docking device 240 can, for example, be used as the docking device 52 in a prosthetic valve implantation procedure, as described above with reference to FIGS. 1-4. As depicted in FIG. 10, the docking device in its deployed configuration can be configured to receive and secure a prosthetic valve within the docking device, thereby securing the prosthetic valve at the native valve annulus.

The docking device 240 can comprise a coil 242 and a guard member 244 covering at least a portion of the coil 242. In some examples, the coil 242 can include a shape memory material (e.g., nickel titanium alloy or “Nitinol”) such that the docking device 240 (and the coil 242) can move from a substantially straight configuration (or delivery configuration) when disposed within the delivery sheath 204 of the delivery apparatus 200 to a helical, deployed configuration after being removed from the delivery sheath 204.

The coil 242 has a proximal end 242p and a distal end 242d (which also respectively define the proximal and distal ends of the docking device 240). When being disposed within the delivery sheath 204 (e.g., during delivery of the docking device 240 into the vasculature of a patient), a body of the coil 242 between the proximal end 242p and distal end 242d can form a generally straight delivery configuration (i.e., without any coiled or looped portions, but can be flexed or bent) so as to maintain a small radial profile when moving through a patient's vasculature. After being removed from the delivery sheath 204 and deployed at an implant position, the coil 242 can move from the delivery configuration to the helical deployed configuration and wrap around native tissue adjacent the implant position. For example, when implanting the docking device at the location of a native valve, the coil 242 can be configured to surround native leaflets of the native valve (and the chordae tendineae that connects native leaflets to adjacent papillary muscles, if present).

The docking device 240 can be releasably coupled to the delivery apparatus 200. For example, in certain examples, the docking device 240 can be coupled to a delivery apparatus (as described above) via a release suture that can be configured to be tied to the docking device 240 and cut for removal.

As shown in FIG. 10, the coil 242 in the deployed configuration can include a leading turn 246 (or “leading coil”), a central region 248, and a stabilization turn 250 (or “stabilization coil”) around a central longitudinal axis. The central region 248 can possess one or more helical turns having substantially equal inner diameters. The leading turn 246 can extend from a distal end of the central region 248 and has a diameter greater than the diameter of the central region 248, in the illustrated example. The stabilization turn 250 can extend from a proximal end of the central region 248 and has a diameter greater than the diameter of the central region 248, in the illustrated example. In some examples, the stabilization turn 250 can be omitted from the coil 242, for example, when a retention member (such as any retention member described herein) is used to stabilize the positioning of the docking device 240 relative to the native anatomy during an implant procedure. Alternatively, the stabilization turn 250 can have a diameter that is equal, approximately equal, or less than the diameter of the central region 248 (as opposed to larger), and/or the stabilization turn can comprise less of a full turn than depicted in FIG. 10.

Further details of the docking device and its variants are described in International Application No. PCT/US2021/056150, which is incorporated by reference herein in its entirety.

FIG. 11 illustrates a prosthetic heart valve delivery apparatus 300 (which can also be referred to here as an “implant catheter”) that can be used to implant an expandable prosthetic heart valve, according to one example. In some examples, the delivery apparatus 300 is specifically adapted for use in introducing a prosthetic heart valve into a heart. For example, the delivery apparatus 300 can be used as the prosthetic heart valve delivery apparatus 60 in a prosthetic valve implantation procedure, as described above with reference to FIG. 3A.

The delivery apparatus 300 in the illustrated example of FIG. 11 is a balloon catheter comprising a handle 302 and a steerable, outer shaft 304 extending distally from the handle 302. The delivery apparatus 300 can further comprise an intermediate shaft 306 (which also may be referred to as a balloon shaft) that extends proximally from the handle 302 and distally from the handle 302, the portion extending distally from the handle 302 also extending coaxially through the outer shaft 304. In some examples, the delivery apparatus 300 can further comprise an inner shaft extending distally from the handle 302 coaxially through the intermediate shaft 306 and the outer shaft 304 and proximally from the handle 302 coaxially through the intermediate shaft.

The outer shaft 304 and the intermediate shaft 306 can be configured to translate (e.g., move) longitudinally, along a central longitudinal axis 320 of the delivery apparatus 300, relative to one another to facilitate delivery and positioning of a prosthetic valve at an implantation site in a patient's body.

The intermediate shaft 306 can include a proximal end portion that extends proximally from a proximal end of the handle 302, to an adaptor 312. The adaptor 312 can include a first port 338 configured to receive a guidewire therethrough and a second port 340 configured to receive fluid (e.g., inflation fluid) from a fluid source. The second port 340 can be fluidly coupled to an inner lumen of the intermediate shaft 306.

In some examples, the intermediate shaft 306 can further include a distal end portion that extends distally beyond a distal end of the outer shaft 304 when a distal end of the outer shaft 304 is positioned away from an inflatable balloon 318 of the delivery apparatus 300. A distal end portion of the inner shaft can extend distally beyond the distal end portion of the intermediate shaft 306 toward or to a nose cone 322 at a distal end of the delivery apparatus 300.

In some examples, a distal end of the balloon 318 can be coupled to a distal end of the delivery apparatus 300, such as to the nose cone 322 (as shown in FIG. 11), or to an alternate component at the distal end of the delivery apparatus 300 (e.g., a distal shoulder). An intermediate portion of the balloon 318 can overlay a valve mounting portion 324 of a distal end portion of the delivery apparatus 300 and a distal end portion of the balloon 318 can overly a distal shoulder of the delivery apparatus 300. As shown in FIG. 11, a prosthetic heart valve 350 can be mounted around the balloon 318, at the valve mounting portion 324 of the delivery apparatus 300, in a radially compressed state. The prosthetic heart valve 350 can be configured to be radially expanded by inflation of the balloon 318 at a native valve annulus, as described above with reference to FIG. 3A.

A balloon shoulder assembly of the delivery apparatus 300, which includes the distal shoulder, is configured to maintain the prosthetic heart valve 350 (or other medical device) at a fixed position on the balloon 318 during delivery through the patient's vasculature.

The outer shaft 304 can include a distal tip portion 328 mounted on its distal end. In some examples, the outer shaft 304 and the intermediate shaft 306 can be translated axially relative to one another to position the distal tip portion 328 adjacent to a proximal end of the valve mounting portion 324, when the prosthetic valve 350 is mounted in the radially compressed state on the valve mounting portion 324 (as shown in FIG. 11) and during delivery of the prosthetic valve to the target implantation site. As such, the distal tip portion 328 can be configured to resist movement of the prosthetic valve 350 relative to the balloon 318 proximally, in the axial direction, relative to the balloon 318, when the distal tip portion 328 is arranged adjacent to a proximal side of the valve mounting portion 324.

An annular space can be defined between an outer surface of the inner shaft and an inner surface of the intermediate shaft 306 and can be configured to receive fluid from a fluid source via the second port 340 of the adaptor 312. The annular space can be fluidly coupled to a fluid passageway formed between the outer surface of the distal end portion of the inner shaft and an inner surface of the balloon 318. As such, fluid from the fluid source can flow to the fluid passageway from the annular space to inflate the balloon 318 and radially expand and deploy the prosthetic valve 350.

An inner lumen of the inner shaft can be configured to receive a guidewire therethrough, for navigating the distal end portion of the delivery apparatus 300 to the target implantation site.

The handle 302 can include a steering mechanism configured to adjust the curvature of the distal end portion of the delivery apparatus 300. In the illustrated example, for example, the handle 302 includes an adjustment member, such as the illustrated rotatable knob 360, which in turn is operatively coupled to the proximal end portion of a pull wire. The pull wire can extend distally from the handle 302 through the outer shaft 304 and has a distal end portion affixed to the outer shaft 304 at or near the distal end of the outer shaft 304. Rotating the knob 360 can increase or decrease the tension in the pull wire, thereby adjusting the curvature of the distal end portion of the delivery apparatus 300. Further details on steering or flex mechanisms for the delivery apparatus can be found in U.S. Pat. No. 9,339,384, as previously incorporated by reference above.

The handle 302 can further include an adjustment mechanism 361 including an adjustment member, such as the illustrated rotatable knob 362, and an associated locking mechanism including another adjustment member, configured as a rotatable knob 378. The adjustment mechanism 361 is configured to adjust the axial position of the intermediate shaft 306 relative to the outer shaft 304 (e.g., for fine positioning at the implantation site).

Prosthetic valves disclosed herein (e.g., prosthetic heart valve 350, prosthetic heart valve 62, etc.) can be radially compressible and expandable between a radially compressed state and a radially expanded state. Thus, the prosthetic valves can be crimped on or retained by an implant delivery apparatus (e.g., delivery apparatus 300, delivery apparatus 60, etc.) in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

FIG. 12 illustrates the prosthetic valve 350 in a radially expanded position. The prosthetic valve 350 can be used as the prosthetic heart valve 62 in a prosthetic valve implantation procedure, as described above with reference to FIGS. 1-4. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device (e.g., docking device 240, etc.) that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein in its entirety. In another example, the disclosed prosthetic valves can be implanted within a docking device implanted within or at the native mitral valve, such as disclosed in PCT Publication No. WO2020/247907, which is incorporated by reference herein in its entirety. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated by reference herein in its entirety.

The prosthetic valve 350 can be used as the prosthetic heart valve 62 in a prosthetic valve implantation procedure, as described above with reference to FIGS. 1-4. As shown in FIG. 12, the prosthetic valve 350 can include a frame 352 and a plurality of leaflets 354 can be situated at least partially within the frame 352. The prosthetic valve 350 can also include an outer covering 356 situated about the frame 352. As shown in FIG. 12, the prosthetic valve 350 includes an inflow end 357 and an outflow end 358. The terms “inflow” and “outflow” are related to the normal direction of blood flow (e.g., antegrade blood flow) through the prosthetic valve 350. For example, the leaflets 354 can allow blood flow through the valve 350 in a direction from the inflow end 357 to the outflow end 358 and prevent the reverse flow (e.g., prevent flow in a direction from the outflow end 358 to the inflow end 357).

The frame 352 can be made of any of various suitable plastically-expandable materials (e.g., stainless steel, etc.) or self-expanding materials (e.g., Nitinol) as known in the art. When constructed of a plastically-expandable material, the frame 352 (and thus the valve 350) can be crimped to a radially compressed state on a delivery catheter and then expanded inside a patient by an inflatable balloon or equivalent expansion mechanism. When constructed of a self-expandable material, the frame 352 (and thus the valve 350) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (e.g., the frame 352) include, metal alloys, polymers, or combinations thereof. Example metal alloys can comprise one or more of the following: nickel, cobalt, chromium, molybdenum, titanium, or other biocompatible metal. In some examples, the frame 352 can comprise stainless steel. In some examples, the frame 352 can comprise cobalt-chromium. In some examples, the frame 352 can comprise nickel-cobalt-chromium. In some examples, the frame 352 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Further details of the prosthetic heart valve and its variants are described in U.S. Pat. No. 11,185,406, which is incorporated by reference herein in its entirety.

As noted above, the delivery apparatus 200 and/or the delivery apparatus 300 can be introduced into a patient's vasculature via a guide catheter, such as the guide catheter 100 of FIGS. 5-8. For example, to introduce the delivery apparatus 200 and/or 300 (or an alternate implant catheter or delivery apparatus) into the vasculature of a patient, the shaft 104 of the guide catheter 100 can first be inserted into the patient's vasculature and navigated through the vasculature toward a target implantation site for a medical device or implant. The handle 102 of the guide catheter 100 remains outside the patient and can be accessed by a user (e.g., a physician). The distal end portion 205 of the delivery apparatus 200 and/or the distal end portion of the delivery apparatus 300 (e.g., the nose cone 322 and radially compressed prosthetic heart valve 350 of the delivery apparatus 300) can then be inserted into the main inlet port 130 of the handle 102 of the guide catheter 100 and pushed through the main lumen 112 of the shaft 104, toward the implantation site.

The example transcatheter heart valve replacement procedure (e.g., a mitral valve replacement procedure) described above in connection with FIGS. 1-4 utilizes the guide catheter 30. As explained below, when a guide catheter (e.g., guide catheter 100, guide catheter 400, etc.) that is compatible with a retention member (e.g., retention member 124, retention member 524, retention member 624, etc.) is used in this procedure, the procedure can include several additional and/or alternate steps to those described above in connection with FIGS. 1-4. The following example procedure uses guide catheter 100, retention member 124, docking device 240, delivery apparatus 200, prosthetic heart valve 350 and delivery apparatus 300 as examples, although any of the guide catheters, retention members, delivery apparatuses, docking devices, and prosthetic heart valves described herein can be used in the following procedural steps. As illustrated in FIGS. 13-18, the guide catheter 100 does not include the optional positioning structure 700. For purposes of illustration, the docking device 240 does not include the guard member 244.

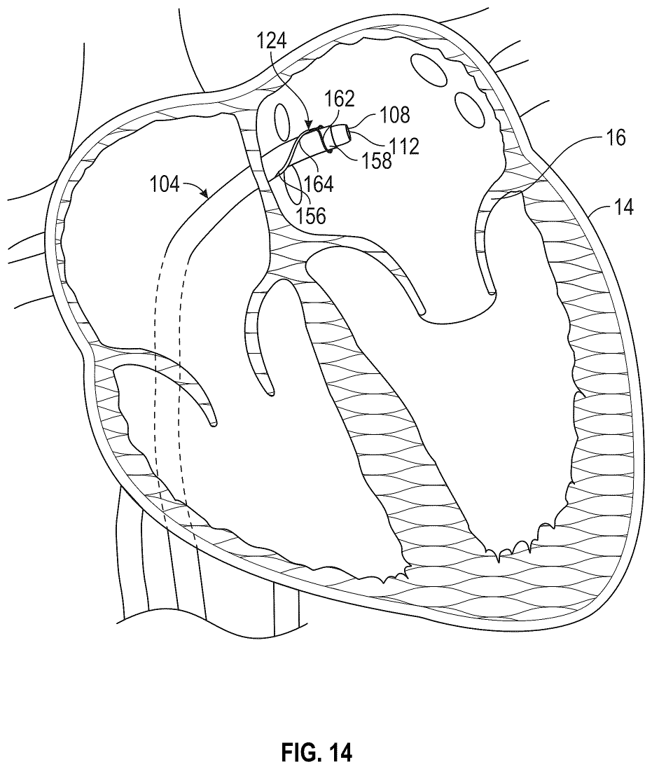

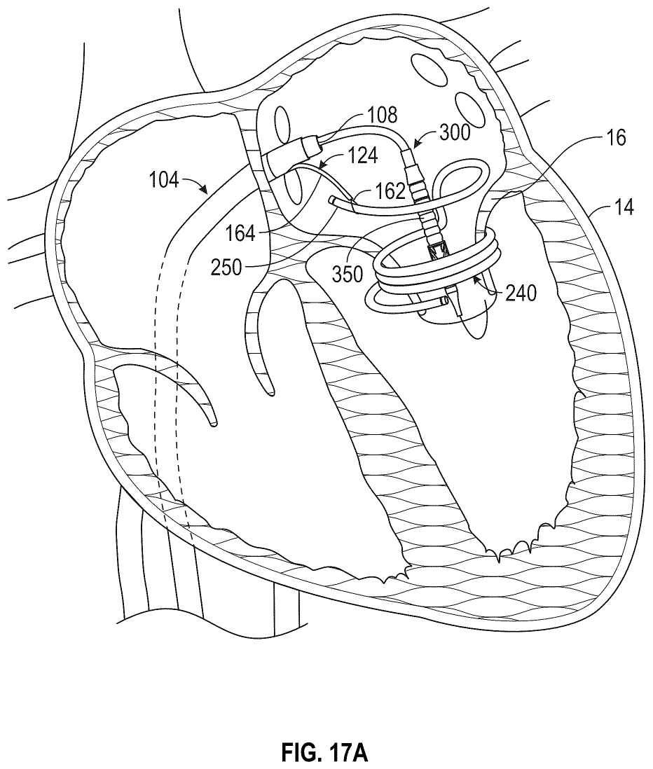

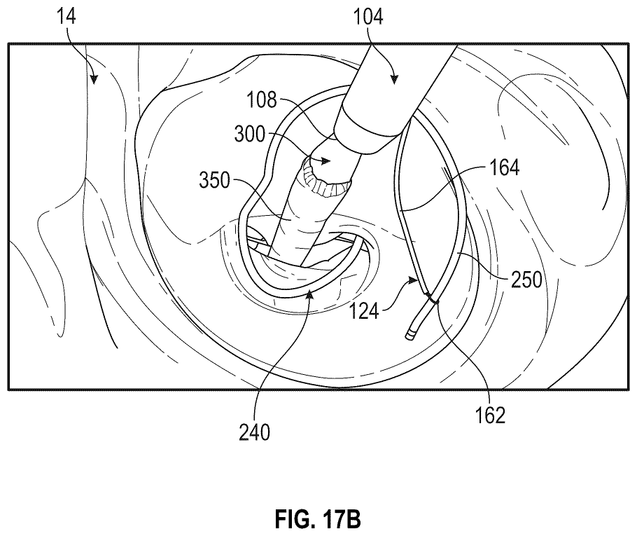

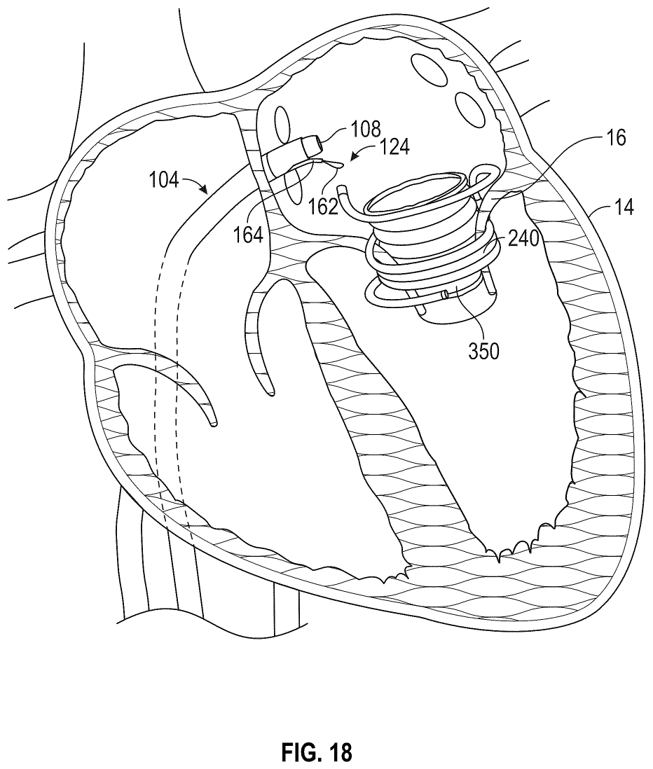

During the procedure, after a user creates a pathway to a patient's native heart valve using the guide catheter 100 (see FIG. 1), the user then distally advances the retention member 124 from an insertion configuration (e.g., external insertion configuration, fully internal configuration, etc.) to a functional configuration (FIGS. 13-14). After the retention member 124 is in the functional configuration (FIG. 14), the user then delivers and implants a docking device 240 at the patient's native heart valve using delivery apparatus 200 (FIG. 15-16B). Specifically, after the docking device 240 is delivered (e.g., through the retention element 162), the user positions the retention element 162 adjacent a portion of the docking device 240 (FIG. 15) and the user actuates the actuation element 164 to couple the retention element 162 to the docking device 240 (FIGS. 16A-B). The retention member 124 remains coupled to the docking device 240 as the docking device 240 is implanted (e.g., as the suture 215 coupling the docking device 240 to the delivery apparatus 200 is removed from the docking device 240) as well as during insertion of the delivery apparatus 300 and implantation of a prosthetic heart valve 350 (FIGS. 17A-B). After the user implants the prosthetic heart valve 350 within the implanted docking device 240 using delivery apparatus 300 (FIGS. 17A-B), the user removes the delivery apparatus 300 from the patient and removes the retention member 124 from the docking device 240 (FIG. 18). Thereafter, the user removes the guide catheter 100 and the retention member 124 from the patient (see FIG. 4).

Referring again to FIG. 13, for a procedure to replace a native mitral valve 16, the guide catheter 100 is positioned such that the distal end 108 of the shaft 104 is within the left atrium 18 of the heart 14. Specifically, the guide catheter 100 is advanced such that the minor lumen exit 156 is disposed within the left atrium 18. The distance D (FIG. 7) between the exit of the main lumen 112 (the distal end 108) and the minor lumen exit 156 is sized such that the minor lumen exit 156 and the exit of the main lumen 112 can both be located within a target zone for implantation (e.g., both within the left atrium 18, etc.). For example, the distance D can be less than 10 cm to enable both the exit of the main lumen 112 and the minor lumen exit 156 to be disposed within the left atrium 18 simultaneously. In some examples, the distance D is approximately 2 cm. In some examples, although not shown, the distance D is 0 cm, such that the exit of the main lumen and the distal-most portion of the minor lumen exit 156 are aligned. In examples where both lumen exits are not within the left atrium 18 (not shown), operation of the retention member 124 may result in unnecessary friction and/or damage to the atrial septum 22. An appropriate distance D also enables the retention member 124 to be operated such that the docking device 240 can be manipulated by the retention member 124 relative to the guide catheter 100 at a desired angle. As shown, advancing the guide catheter 100 such that the minor lumen exit 156 has passed through the atrial septum 22 and is positioned within the left atrium 18 also enables at least a portion of the flex region 128 to be disposed within the left atrium 18.

As shown in FIG. 13, the retention member 124 has been distally advanced from an insertion configuration (e.g., an external insertion configuration, a fully internal insertion configuration, etc.) to a position where the retention element 162 and the actuation element 164 are at least partially distal to the minor lumen exit 156 of the guide catheter 100 (e.g., at least partially external to the minor lumen 116). The position shown in FIG. 13 enables the retention member 124 is an intermediate step between the insertion configuration and a functional configuration of the retention member 124. FIG. 14 illustrates the retention member 124 in the functional configuration where the retention element 162 is secured to the outer surface 158 of the shaft 104. Specifically, the actuation element 164 is actuated such that the retention element 162 is in a tensioned state around the outer surface 158 of the shaft 104. In the functional configuration, the retention element 162 is distal to the minor lumen exit 156.

In general, the functional configuration is a position of a retention member that allows the retention member to releasably couple to a prosthetic medical device (e.g., the docking device 240). In some examples, the functional configuration simplifies or streamlines the coupling of the retention member and the prosthetic medical device. For example, the functional configuration for the retention member 124 (FIG. 14) enables the delivery apparatus 200 (therefore, the docking device 240) to pass through the opening of the retention element 162 as the delivery apparatus 200 is advanced from the main lumen 112 of the guide catheter 100. In this way, the retention element 162 does not need to be otherwise manipulated to position the docking device 240 within the retention element 162. That is, the retention element 162 surrounds the docking device 240 and only needs to be actuated by the actuation element 164 in order for the retention member 124 to be coupled to the docking device 240. Accordingly, advancing or deploying the docking device 240 positions the docking device 240 in the correct position relative to the retention member 124 for coupling the retention member to the docking device 240. This simplifies the procedure for the user as the user can surround a portion of the docking device 240 with the retention element 162 (e.g., after the docking device 240 is deployed) with fewer manipulations of the retention element 162.

In some examples, the retention member 124 can be disposed in the functional configuration during insertion of the guide catheter 100. That is, for some retention members, the insertion configuration of a retention member can be the same as the functional configuration of that retention member. Specifically, for retention member 124, the partially internal insertion configuration (FIG. 8) is the same as the functional configuration (FIG. 14). As such, in some examples, the retention member 124 can be positioned in the partially internal insertion configuration, and therefore the functional configuration, relative to the guide catheter 100 during insertion of the guide catheter 100. In these examples, the intermediate step of advancing the retention member 124 distally from the minor lumen exit 156 shown in FIG. 13 can be omitted.

The functional configuration can be different for different retention members. For example, retention members 524, 624 can be in the functional configuration while fully disposed within the minor lumen 116. Specifically, because the retention elements 562, 662 do not include a loop or lasso, the retention elements 562, 662 are able to grip or grasp a docking device 240 regardless of whether the delivery apparatus 200 and docking device 240 are advanced through the retention element 562, 662. Rather, the retention member 524, 624 are simply advanced from the minor lumen exit 156 and positioned adjacent to the docking device 240 to couple the retention member 524, 624 to the docking device 240. Specifically, the actuation element 564, 664 is actuated to couple the retention element 562, 662 to the docking device 240.

The user positions the retention member 124 in the functional configuration (FIG. 14) prior to delivering and implanting the docking device 240 at the patient's native mitral valve 16 using a docking device delivery apparatus 200 (FIGS. 15-16B). By positioning the retention member 124 in the functional configuration (FIG. 14), the delivery apparatus 200 (and therefore docking device 240) can be advanced through the loop 162 of the retention member 124, as described above.

FIGS. 15-16B illustrate the docking device 240 as deployed from to the delivery apparatus 200. Specifically, the docking device 240 is advanced from the delivery apparatus 200 in a distal direction, such that the docking device 240 is deployed at the target location (e.g., native mitral valve 16). As shown, the docking device 240 can be coupled to the delivery apparatus 200 via release suture 215.

The retention member 124 is transitioned from the tensioned state around the outer surface 158 of the shaft 104 (FIG. 14) to a relaxed state using the actuation element 164, such that the retention member 124 can be re-positioned around a prosthetic implant (e.g., the docking device 240). For example, as shown in FIG. 15, the retention member 124 is positioned such that the retention element 162 is disposed around a portion of the docking device 240 (e.g., a proximal portion of the docking device 240). In some examples, as shown, the retention element 162 is disposed around the stabilizing turn 250 of the docking device 240. In some examples, the retention element 162 can be disposed around other portions of the docking device 240.

To move the retention member 124 from the functional configuration (FIG. 14) to the positioning shown in FIG. 15, the user actuates the actuation element 164 to loosen the retention element 162 from the outer surface 158 of the shaft 104 (e.g., translates the outer shaft 164 proximally relative to the loop 162, etc.) to the relaxed state. Subsequently, the user advances the retention member 124 distally until the retention element 162 is positioned in the desired location (e.g., around the portion of the docking device 240).

As shown in FIGS. 16A-16B, after the retention element 162 is positioned in the desired location, the user then actuates the actuation element 164 to tighten or tension the retention element 162 around the portion of the docking device 240, such that the retention member 124 is coupled to the docking device 240 (e.g., in the tensioned state). When coupled, the user can remove (e.g., cut) the suture 215 and remove the delivery apparatus 200 from the patient.

Then, as shown in FIGS. 17A-17B, with the retention member 124 coupled to the docking device 240, the user can insert the delivery apparatus 300 through the main lumen 112 of the guide catheter 100 such that the prosthetic heart valve 350 (e.g., as coupled to the delivery apparatus 300) is positioned within the docking device 240. Once positioned, the user can deploy the prosthetic heart valve 350 within the docking device 240 using the delivery apparatus 300.

Thereafter, as shown in FIG. 18, the delivery apparatus 300 can be retracted through the main lumen of the guide catheter 100 and out of the patient. The user can then remove or decouple the retention member 124 from the docking device 240 using actuation element 164. Subsequently, the user can remove the guide catheter 100 and the retention member 124 from the patient (see FIG. 4).

After implanting the docking device 240, during removal of the delivery apparatus 200, and during insertion of a prosthetic valve delivery apparatus (e.g., delivery apparatus 300), the retention member 124 remains coupled to the docking device 240. In some examples, the retention member 124 can be prevented from moving relative to the guide catheter 100 during these (and other) steps. For example, the user can operate lock 140 to limit and/or prevent movement of the retention member 124 relative to the minor lumen 116 (e.g., during insertion of the guide catheter 100 into the heart 14, during removal of the delivery apparatus 200, during insertion and removal of the delivery apparatus 300, etc.). It should be appreciated that the user has the ability to position or re-position the docking device 240 relative to the native anatomy of the heart 14 as needed during these (and other) steps (e.g., steps where the retention member 124 is coupled to the docking device 240). For example, the user can manipulate the retention member 124 when coupled to the docking device 240 to position or re-position the docking device 240 relative to the native anatomy of the heart 14 (e.g., relative to the native mitral valve 16).

As a result, the positioning of an implantable prosthetic medical device can be easily maintained and/or adjusted during an implantation procedure in a streamlined manner. In some examples, this can increase an efficiency of a prosthetic device implantation procedure.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus comprising: a handle; a shaft extending distally from the handle and having a longitudinal axis, a distal end, and a proximal end, the shaft comprising a first inner surface defining a main lumen and a second inner surface defining a minor lumen; and a retention member partially disposed within the minor lumen, the retention member comprising a retention element and an actuation element, wherein the retention member is selectively axially movable relative to the minor lumen and configured to releasably couple to a prosthetic implant at a target location.

Example 2. The delivery apparatus of any example herein, particularly example 1, wherein the retention element comprises one of: a snare, a loop, a lasso, claws, talons, teeth, barbs, and prongs.

Example 3. The delivery apparatus of any example herein, particularly either example 1 or example 2, wherein the actuation element comprises one of: an outer shaft, a pull shaft, and a pull wire.

Example 4. The delivery apparatus of any example herein, particularly any one of examples 1-3, wherein the shaft further comprises at least one third inner surface defining at least one pull wire lumen, wherein a pull wire is disposed within each pull wire lumen.

Example 5. The delivery apparatus of any example herein, particularly example 4, wherein the main lumen is coaxial with the longitudinal axis, wherein the pull wire lumen is a first radial distance from the longitudinal axis, wherein the minor lumen is a second radial distance from the longitudinal axis, wherein the second radial distance is greater than the first radial distance.

Example 6. The delivery apparatus of any example herein, particularly either example 4 or example 5, wherein the shaft comprises two third inner surfaces defining two pull wire lumens, wherein a center of the minor lumen is circumferentially between the two pull wire lumens.

Example 7. The delivery apparatus of any example herein, particularly any one of examples 1-6, wherein the shaft further defines a minor lumen exit coupled to the minor lumen, wherein the minor lumen exit comprises a distal surface.

Example 8. The delivery apparatus of any example herein, particularly example 7, wherein the main lumen extends axially from the distal end to the proximal end of the shaft, and wherein the minor lumen extends proximally from the minor lumen exit.

Example 9. The delivery apparatus of any example herein, particularly either example 7 or example 8, wherein the distal surface is offset axially from the distal end of the shaft by a distance D.

Example 10. The delivery apparatus of any example herein, particularly example 9, wherein the distance D is less than 10 centimeters.

Example 11. The delivery apparatus of any example herein, particularly any one of examples 7-10, wherein the distal surface is angled relative to the longitudinal axis.

Example 12. The delivery apparatus of any example herein, particularly any one of examples 7-11, further comprising a locking mechanism coupled to a proximal portion of the minor lumen, the locking mechanism configured to selectively lock movement of the retention member relative to the minor lumen.

Example 13. The delivery apparatus of any example herein, particularly example 12, wherein the locking mechanism comprises a tuohy borst adapter.

Example 14. The delivery apparatus of any example herein, particularly any one of examples 7-13, wherein the shaft further comprises a positioning structure distal to the minor lumen exit, wherein the positioning structure comprises a notch.

Example 15. The delivery apparatus of any example herein, particularly example 14, wherein the retention element is at least partially disposed within the notch.

Example 16. The delivery apparatus of any example herein, particularly either example 14 or example 15, wherein the notch is a grooved notch.

Example 17. The delivery apparatus of any example herein, particularly any one of examples 14-16, wherein the positioning structure further comprises a projection extending radially outwards from the shaft, wherein the projection is distal to the notch.

Example 18. The delivery apparatus of any example herein, particularly example 17, wherein the projection has a distal surface that is angled relative to the longitudinal axis.

Example 19. The delivery apparatus of any example herein, particularly either example 17 or example 18, wherein the projection is circumferentially aligned with the minor lumen exit.

Example 20. The delivery apparatus of any example herein, particularly any one of examples 7-19, further comprising a reinforcing material disposed circumferentially around the main lumen and the minor lumen, wherein a distal end of the reinforcing material is proximal to the minor lumen exit.

Example 21. The delivery apparatus of any example herein, particularly any one of examples 7-20, wherein the shaft comprises a flex region, wherein the minor lumen exit is disposed within the flex region.

Example 22. The delivery apparatus of any example herein, particularly any one of examples 1-21, further comprising a connector shaft coupled to the minor lumen adjacent to the proximal end of the shaft, the connector shaft angled relative to the longitudinal axis.

Example 23. The delivery apparatus of any example herein, particularly any one of examples 1-22, wherein the shaft comprises an outer surface, wherein the retention element is partially external to the minor lumen and contacts the outer surface of the shaft.

Example 24. A delivery apparatus comprising: a handle; and a shaft extending distally from the handle and including a distal end and a proximal end, the shaft comprising a first inner surface defining a main lumen, a second inner surface defining a minor lumen, and a third inner surface defining a pull wire lumen, the shaft including a longitudinal axis that is coaxial with the main lumen; wherein the pull wire lumen is a first radial distance from the longitudinal axis, wherein the minor lumen is a second radial distance from the longitudinal axis, wherein the second radial distance is greater than the first radial distance.

Example 25. The delivery apparatus of any example herein, particularly any example herein, particularly example 24, further comprising a pull wire disposed within the pull wire lumen.

Example 26. The delivery apparatus of any example herein, particularly any example herein, particularly either example 24 or example 25, wherein the shaft comprises two third inner surfaces defining two pull wire lumens, wherein a center of the minor lumen is circumferentially between the two pull wire lumens.

Example 27. The delivery apparatus of any example herein, particularly any one of examples 24-26, further comprising a retention member at least partially disposed within the minor lumen, wherein the retention member is selectively axially movable relative to the minor lumen and configured to releasably couple to a prosthetic implant at a target location.

Example 28. The delivery apparatus of any example herein, particularly example 27, wherein the retention member comprises an outer shaft and a snare disposed at least partially within the outer shaft, the outer shaft slidable relative to the snare to actuate the snare.

Example 29. The delivery apparatus of any example herein, particularly example 28, wherein the shaft comprises an outer surface, wherein the snare is partially external to the minor lumen and contacts the outer surface of the shaft.

Example 30. The delivery apparatus of any example herein, particularly any one of examples 27-29, wherein the shaft further defines a minor lumen exit coupled to the minor lumen, wherein the minor lumen exit comprises a distal surface.

Example 31. The delivery apparatus of any example herein, particularly example 30, wherein the main lumen extends axially from the distal end to the proximal end of the shaft, wherein the minor lumen extends proximally from the minor lumen exit, wherein the minor lumen exit axially offset from the distal end of the shaft.

Example 32. The delivery apparatus of any example herein, particularly either example 30 or example 31, wherein the distal surface is angled relative to the longitudinal axis.

Example 33. The delivery apparatus of any example herein, particularly any one of examples 30-32, further comprising a reinforcing material disposed circumferentially around the main lumen and the minor lumen, wherein a distal end of the reinforcing material is proximal to the minor lumen exit.

Example 34. The delivery apparatus of any example herein, particularly any one of examples 30-33, wherein the shaft further comprises a positioning structure distal to the minor lumen exit, wherein the positioning structure comprises a notch.

Example 35. The delivery apparatus of any example herein, particularly example 34, wherein a portion of the retention member is at least partially disposed within the notch.

Example 36. The delivery apparatus of any example herein, particularly either example 34 or example 35, wherein the notch is a grooved notch.

Example 37. The delivery apparatus of any example herein, particularly any one of examples 34-36, wherein the positioning structure further comprises a projection extending radially outwards from the shaft, wherein the projection is distal to the notch.

Example 38. The delivery apparatus of any example herein, particularly example 37, wherein the projection has a distal surface that is angled relative to the longitudinal axis.

Example 39. The delivery apparatus of any example herein, particularly either example 37 or example 38, wherein the projection is circumferentially aligned with the minor lumen exit.

Example 40. A delivery apparatus comprising: a handle; and a shaft extending distally from the handle, the shaft having a longitudinal axis and multiple embedded lumens that are parallel to the longitudinal axis, the multiple embedded lumens including a main lumen, a minor lumen, and at least two pull wire lumens, wherein a center of the minor lumen is circumferentially between the at least two pull wire lumens.

Example 41. The delivery apparatus of any example herein, particularly example 40, further comprising a pull wire disposed within each of the pull wire lumens.

Example 42. The delivery apparatus of any example herein, particularly either example 40 or example 41, wherein each pull wire lumen is a first radial distance from the main lumen, wherein the minor lumen is a second radial distance from the main lumen, wherein the second radial distance is greater than the first radial distance.

Example 43. The delivery apparatus of any example herein, particularly any one of examples 40-42, wherein the shaft further defines a main lumen exit coupled to the main lumen and a minor lumen exit coupled to the minor lumen, wherein the minor lumen exit is offset axially from the main lumen exit by a distance D.

Example 44. The delivery apparatus of any example herein, particularly example 43, wherein the distance D is less than 10 centimeters.

Example 45. The delivery apparatus of any example herein, particularly either example 43 or example 44, wherein the minor lumen exit comprises a distal surface that is angled relative to the main lumen by an angle α.

Example 46. The delivery apparatus of any example herein, particularly example 45, wherein the angle α is less than 90 degrees.

Example 47. The delivery apparatus of any example herein, particularly any one of examples 43-46, wherein the shaft further comprises a positioning structure distal to the minor lumen exit, wherein the positioning structure comprises a notch.

Example 48. The delivery apparatus of any example herein, particularly example 47, further comprising a retention member at least partially disposed within the minor lumen, wherein the retention member is selectively axially movable relative to the minor lumen and configured to releasably couple to a prosthetic implant at a target location.

Example 49. The delivery apparatus of any example herein, particularly example 48, wherein the retention member comprises a retention element and an actuation element.

Example 50. The delivery apparatus of any example herein, particularly example 49, wherein the shaft comprises an outer surface, wherein the retention element is partially external to the minor lumen and contacts the outer surface of the shaft.

Example 51. The delivery apparatus of any example herein, particularly either example 49 or example 50, wherein the retention element is at least partially disposed within the notch.

Example 52. The delivery apparatus of any example herein, particularly any one of examples 47-51, wherein the positioning structure further comprises a projection extending radially outwards from the shaft, wherein the projection is distal to the notch.

Example 53. The delivery apparatus of any example herein, particularly example 52, wherein the projection has a distal surface that is angled relative to the longitudinal axis.

Example 54. The delivery apparatus of any example herein, particularly either example 52 or example 53, wherein the projection is circumferentially aligned with the minor lumen exit.

Example 55. The delivery apparatus of any example herein, particularly any one of examples 43-54, further comprising a reinforcing material disposed circumferentially around the main lumen and the minor lumen, wherein a distal end of the reinforcing material is proximal to the minor lumen exit.

Example 56. The delivery apparatus of any example herein, particularly any one of examples 43-55, wherein the shaft comprises a flex region, wherein the minor lumen exit is disposed within the flex region.

Example 57. A delivery apparatus comprising: a handle; a shaft having a distal end, a proximal end, and a longitudinal axis extending between the distal end and the proximal end, the shaft comprising: a first inner surface defining a main lumen, the main lumen coaxial with the longitudinal axis; a second inner surface defining a minor lumen; and a minor lumen exit coupled to the minor lumen, wherein the minor lumen exit comprises a distal surface that is angled relative to the longitudinal axis.

Example 58. The delivery apparatus of any example herein, particularly example 57, wherein the minor lumen exit is axially offset from the distal end of the shaft by a distance D.

Example 59. The delivery apparatus of any example herein, particularly example 58, wherein the distance D is less than 10 centimeters.

Example 60. The delivery apparatus of any example herein, particularly example 59, wherein the distance D is approximately 2 centimeters.

Example 61. The delivery apparatus of any example herein, particularly any one of examples 57-60, wherein the distal surface is angled relative to the longitudinal axis by an angle α, wherein the angle α is less than 90 degrees.

Example 62. The delivery apparatus of any example herein, particularly example 61, wherein the angle α is approximately 20 degrees.

Example 63. The delivery apparatus of any example herein, particularly any one of examples 57-62, further comprising a retention member at least partially disposed within the minor lumen, the retention member comprising a retention element and an actuation element, wherein the retention member is selectively axially movable relative to the minor lumen and configured to releasably couple to a prosthetic implant at a target location.

Example 64. The delivery apparatus of any example herein, particularly example 63, wherein the retention element comprises one of: a snare, a loop, a lasso, claws, talons, teeth, barbs, and prongs.

Example 65. The delivery apparatus of any example herein, particularly either example 63 or example 64, wherein the actuation element comprises one of: an outer shaft, a pull shaft, and a pull wire.

Example 66. The delivery apparatus of any example herein, particularly any one of examples 63-65, wherein the shaft comprises an outer surface, wherein the retention element is partially external to the minor lumen and contacts the outer surface of the shaft.

Example 67. The delivery apparatus of any example herein, particularly any one of examples 57-66, wherein the shaft further comprises at least one third inner surface defining at least one pull wire lumen, wherein a pull wire is disposed within each pull wire lumen.

Example 68. The delivery apparatus of any example herein, particularly example 67, wherein the pull wire lumen is a first radial distance from the longitudinal axis, wherein the minor lumen is a second radial distance from the longitudinal axis, wherein the second radial distance is greater than the first radial distance.

Example 69. The delivery apparatus of any example herein, particularly either example 67 or example 68, wherein the shaft comprises two third inner surfaces defining two pull wire lumens, wherein a center of the minor lumen is circumferentially between the two pull wire lumens.

Example 70. The delivery apparatus of any example herein, particularly any one of examples 57-69, wherein the shaft further comprises a positioning structure distal to the minor lumen exit, wherein the positioning structure comprises a notch.

Example 71. The delivery apparatus of any example herein, particularly example 70, wherein the notch is a grooved notch.

Example 72. The delivery apparatus of any example herein, particularly either example 70 or example 71, wherein the positioning structure further comprises a projection extending radially outwards from the shaft, wherein the projection is distal to the notch.

Example 73. The delivery apparatus of any example herein, particularly example 72, wherein the projection has a distal surface that is angled relative to the longitudinal axis.

Example 74. The delivery apparatus of any example herein, particularly example 73, wherein the distal surface of the projection is angled relative to the longitudinal axis by less than 90 degrees.

Example 75. The delivery apparatus of any example herein, particularly either example 73 or example 74, wherein the distal surface of the projection is angled relative to the longitudinal axis by approximately 20 degrees.

Example 76. The delivery apparatus of any example herein, particularly any one of examples 72-75, wherein the projection is circumferentially aligned with the minor lumen exit.

Example 77. The delivery apparatus of any example herein, particularly any one of examples 57-76, wherein the shaft comprises a flex region, wherein the minor lumen exit is disposed within the flex region.

Example 78. A delivery apparatus comprising: a handle; and a shaft extending distally from the handle and including an outer surface, a distal end, a proximal end and a longitudinal axis, the shaft comprising a first lumen that is coaxial with the longitudinal axis, a second lumen, and a positioning structure, the first lumen extending axially between the distal end and the proximal end, the second lumen having an exit that is axially offset from the distal end, the positioning structure disposed distal to the exit of the second lumen, the positioning structure comprising a notch.

Example 79. The delivery apparatus of any example herein, particularly example 78, wherein the first lumen extends axially from the distal end to the proximal end of the shaft, and wherein the second lumen extends proximally from the exit.

Example 80. The delivery apparatus of any example herein, particularly either example 78 or example 79, wherein the exit comprises a distal surface, wherein the distal surface is offset axially from the distal end of the shaft by a distance of less than 10 centimeters.

Example 81. The delivery apparatus of any example herein, particularly example 80, wherein the distal surface is angled relative to the longitudinal axis.

Example 82. The delivery apparatus of any example herein, particularly any one of examples 78-81, wherein the notch is a grooved notch.

Example 83. The delivery apparatus of any example herein, particularly any one of examples 78-82, wherein the positioning structure further comprises a projection extending radially outwards from the shaft, wherein the projection is distal to the notch.

Example 84. The delivery apparatus of any example herein, particularly example 83, wherein the projection has a distal surface that is angled relative to the longitudinal axis.

Example 85. The delivery apparatus of any example herein, particularly either example 83 or example 84, wherein the projection is circumferentially aligned with the exit.

Example 86. The delivery apparatus of any example herein, particularly any one of examples 78-85, further comprising a retention member partially disposed within the second lumen, the retention member comprising a retention element and an actuation element, wherein the retention member is selectively axially movable relative to the second lumen and configured to releasably couple to a prosthetic implant at a target location.

Example 87. The delivery apparatus of any example herein, particularly example 86, wherein the retention element comprises a loop, and wherein the actuation element comprises an outer shaft.

Example 88. The delivery apparatus of any example herein, particularly either example 86 or example 87, wherein the retention element is at least partially disposed within the notch.

Example 89. The delivery apparatus of any example herein, particularly any one of examples 86-88, wherein the shaft comprises an outer surface, wherein the retention element is partially external to the second lumen and contacts the outer surface of the shaft.

Example 90. The delivery apparatus of any example herein, particularly any one of examples 78-89, further comprising a reinforcing material disposed circumferentially around the first lumen and the second lumen, wherein a distal end of the reinforcing material is proximal to the exit.

Example 91. The delivery apparatus of any example herein, particularly any one of examples 78-90, wherein the shaft comprises a flex region, wherein the exit is disposed within the flex region.

Example 92. The delivery apparatus of any example herein, particularly any one of examples 78-91, wherein the shaft further comprises at least one third inner surface defining at least one pull wire lumen, wherein a pull wire is disposed within each pull wire lumen.

Example 93. The delivery apparatus of any example herein, particularly example 92, wherein the pull wire lumen is a first radial distance from the longitudinal axis, wherein the second lumen is a second radial distance from the longitudinal axis, wherein the second radial distance is greater than the first radial distance.

Example 94. The delivery apparatus of any example herein, particularly either example 92 or example 93, wherein the shaft comprises two third inner surfaces defining two pull wire lumens, wherein a center of the second lumen is circumferentially between the two pull wire lumens.

Example 95. A delivery apparatus comprising: a handle; a shaft extending distally from the handle and including an outer surface and a longitudinal axis, the shaft comprising a main lumen and a minor lumen; and a retention member partially disposed within the minor lumen, the retention member comprising a retention element and an actuation element, wherein the retention element is partially external to the minor lumen and contacts the outer surface of the shaft.

Example 96. The delivery apparatus of any example herein, particularly example 95, wherein the retention member is selectively axially movable relative to the minor lumen and configured to releasably couple to a prosthetic implant at a target location.

Example 97. The delivery apparatus of any example herein, particularly either example 95 or example 96, wherein the retention element comprises one of: a snare, a loop, and a lasso.

Example 98. The delivery apparatus of any example herein, particularly any one of examples 95-97, wherein the actuation element comprises an outer shaft that is axially movable relative to the retention element, wherein the outer shaft is configured to transition the retention element between a tensioned state and a relaxed state.

Example 99. The delivery apparatus of any example herein, particularly example 98, wherein the retention element is in the tensioned state when the retention element contacts the outer surface of the shaft.

Example 100. The delivery apparatus of any example herein, particularly any one of examples 95-99, wherein the shaft further comprises at least one pull wire lumen, wherein a pull wire is disposed within each pull wire lumen.

Example 101. The delivery apparatus of any example herein, particularly example 100, wherein the pull wire lumen is a first radial distance from the main lumen, wherein the minor lumen is a second radial distance from the main lumen, wherein the second radial distance is greater than the first radial distance.

Example 102. The delivery apparatus of any example herein, particularly either example 100 or example 101, wherein the shaft comprises two pull wire lumens, wherein a center of the minor lumen is circumferentially between the two pull wire lumens.

Example 103. The delivery apparatus of any example herein, particularly any one of examples 95-102, wherein the shaft further defines a minor lumen exit coupled to the minor lumen, wherein the minor lumen exit comprises a distal surface.

Example 104. The delivery apparatus of any example herein, particularly example 103, wherein the main lumen extends axially from a distal end to a proximal end of the shaft, wherein the minor lumen extends proximally from the minor lumen exit, wherein the minor lumen exit is adjacent to the distal end of the shaft.

Example 105. The delivery apparatus of any example herein, particularly either example 103 or example 104, wherein the distal surface is offset axially from the distal end of the shaft by a distance D.

Example 106. The delivery apparatus of any example herein, particularly example 105, wherein the distance D is less than 10 centimeters.

Example 107. The delivery apparatus of any example herein, particularly either example 105 or example 106, wherein the distance D is approximately 2 centimeters.

Example 108. The delivery apparatus of any example herein, particularly any one of examples 105-107, wherein the distal surface is angled relative to the longitudinal axis.

Example 109. The delivery apparatus of any example herein, particularly any one of examples 105-108, wherein the shaft further comprises a positioning structure distal to the minor lumen exit, wherein the positioning structure comprises a notch.

Example 110. The delivery apparatus of any example herein, particularly example 109, wherein the retention element is at least partially disposed within the notch.

Example 111. The delivery apparatus of any example herein, particularly either example 109 or example 110, wherein the notch is a grooved notch.

Example 112. The delivery apparatus of any example herein, particularly any one of examples 109-111, wherein the positioning structure further comprises a projection extending radially outwards from the shaft, wherein the projection is distal to the notch.

Example 113. The delivery apparatus of any example herein, particularly example 112, wherein the projection has a distal surface that is angled relative to the longitudinal axis.

Example 114. The delivery apparatus of any example herein, particularly either example 112 or example 113, wherein the projection is circumferentially aligned with the minor lumen exit.

Example 115. The delivery apparatus of any example herein, particularly any one of examples 105-114, wherein the shaft comprises a flex region, wherein the minor lumen exit is disposed within the flex region.

Example 116. A delivery assembly comprising: an implant catheter including a prosthetic implant; and a guide catheter comprising: a shaft having a distal end and a proximal end, the shaft including a main lumen configured to receive a portion of the implant catheter therethrough and a minor lumen radially offset from the main lumen; and a retention member at least partially disposed within the minor lumen, the retention member configured to releasably couple to the prosthetic implant.

Example 117. The delivery assembly of example 116, wherein the prosthetic implant is a docking device for a prosthetic heart valve.

Example 118. The delivery assembly of either example 116 or 117, wherein the retention member comprises a retention element and an actuation element.

Example 119. The delivery assembly of example 118, wherein the retention element comprises one of: a snare, a loop, a lasso, claws, talons, teeth, barbs, and prongs.

Example 120. The delivery assembly of either example 118 or example 119, wherein the actuation element comprises one of: an outer shaft, a pull shaft, and a pull wire.

Example 121. The delivery assembly of any one of examples 116-120, wherein the shaft further comprises at least one pull wire lumen, wherein a pull wire is disposed within each pull wire lumen.

Example 122. The delivery assembly of example 121, wherein the pull wire lumen is a first radial distance from the main lumen, wherein the minor lumen is a second radial distance from the main lumen, wherein the second radial distance is greater than the first radial distance.

Example 123. The delivery assembly of either example 121 or example 122, wherein the shaft comprises two pull wire lumens, wherein a center of the minor lumen is circumferentially between the two pull wire lumens.

Example 124. The delivery assembly of any one of examples 116-123, wherein the shaft further defines a minor lumen exit adjacent to the distal end of the shaft and coupled to the minor lumen, wherein the minor lumen exit comprises an angled distal surface.

Example 125. The delivery assembly of example 124, wherein the shaft further comprises a positioning structure distal to the minor lumen exit, wherein the positioning structure comprises a notch and an angled projection distal to the notch.

Example 126. The delivery assembly of any one of examples 116-125, wherein the shaft comprises an outer surface, wherein the retention member is partially external to the minor lumen and at least partially contacts the outer surface of the shaft.

Example 127. A delivery apparatus comprising: a handle; a shaft assembly extending distally from the handle and having a longitudinal axis, a distal end, and a proximal end, the shaft assembly comprising a first shaft defining a main lumen and a second shaft defining a minor lumen, wherein the first and second shafts are coupled together and are parallel to the longitudinal axis; and a retention member partially disposed within the minor lumen, the retention member comprising a retention element and an actuation element, wherein the retention member is selectively axially movable relative to the minor lumen and configured to releasably couple to a prosthetic implant at a target location.

Example 128. The delivery apparatus of any example herein, particularly example 127, wherein the prosthetic implant is a docking device for a prosthetic heart valve.

Example 129. The delivery apparatus of any example herein, particularly either example 127 or 128, wherein the retention member comprises a retention element and an actuation element.

Example 130. The delivery apparatus of any example herein, particularly example 129, wherein the retention element comprises one of: a snare, a loop, a lasso, claws, talons, teeth, barbs, and prongs.

Example 131. The delivery apparatus of any example herein, particularly either example 129 or example 130, wherein the actuation element comprises one of: an outer shaft, a pull shaft, and a pull wire.

Example 132. The delivery apparatus of any example herein, particularly any one of examples 127-131, wherein the shaft assembly further comprises at least one pull wire lumen, wherein a pull wire is disposed within each pull wire lumen.

Example 133. The delivery apparatus of any example herein, particularly example 132, wherein the first shaft includes the at least one pull wire lumen.

Example 134. The delivery apparatus of any example herein, particularly either example 132 or example 133, wherein the shaft assembly comprises two pull wire lumens, wherein the second shaft is coupled to the first shaft at a location circumferentially between the two pull wire lumens.

Example 135. The delivery apparatus of any example herein, particularly any one of examples 127-134, wherein the second shaft defines a minor lumen exit adjacent to the distal end of the first shaft and coupled to the minor lumen, wherein the minor lumen exit comprises a distal surface that is angled relative to the longitudinal axis.

Example 136. The delivery apparatus of any example herein, particularly example 135, wherein the second shaft further comprises a positioning structure distal to the minor lumen exit, wherein the positioning structure comprises a notch and an angled projection distal to the notch.

Example 137. The delivery apparatus of any example herein, particularly any one of examples 127-136, wherein the shaft assembly comprises an outer surface, wherein the retention member is partially external to the minor lumen and at least partially contacts the outer surface of the shaft assembly.

Example 138. A shaft for a delivery apparatus, the shaft comprising: a body having a longitudinal axis, the body comprising: a first inner surface defining a main lumen, the main lumen coaxial with the longitudinal axis; a second inner surface defining a minor lumen; and a third inner surface defining a pull wire lumen, wherein the pull wire lumen is a first radial distance from the longitudinal axis, wherein the minor lumen is a second radial distance from the longitudinal axis, wherein the second radial distance is greater than the first radial distance.

Example 139. A shaft for a delivery apparatus, the shaft comprising: a body having multiple lumens that are embedded within the body, the multiple lumens comprising a main lumen, a minor lumen, and at least two pull wire lumens, wherein the multiple lumens are parallel, wherein a center of the minor lumen is circumferentially between the at least two pull wire lumens.

Example 140. A shaft for a delivery apparatus, the shaft comprising: a body having a distal end, a proximal end, and a longitudinal axis extending between the distal end and the proximal end, the body comprising: a first inner surface defining a main lumen, the main lumen coaxial with the longitudinal axis; a second inner surface defining a minor lumen; and a minor lumen exit adjacent to the distal end of the shaft and coupled to the minor lumen, wherein the minor lumen exit comprises a distal surface that is angled relative to the longitudinal axis.

Example 141. A delivery apparatus comprising: a handle; and the shaft of any one of examples 138-140, the shaft extending distally from the handle.

Example 142. A method for implanting a prosthetic medical device, comprising: inserting a guide catheter into a vessel of a patient; advancing a prosthetic implant coupled to a delivery apparatus through a main lumen of the guide catheter to an implant location; advancing a retention member through a minor lumen of the guide catheter; and coupling the retention member to the prosthetic implant.

Example 143. The method of any example herein, particularly example 142, further comprising releasing the prosthetic implant from the delivery apparatus when the retention member is coupled to the prosthetic implant.

Example 144. The method of any example herein, particularly either example 142 or example 143, wherein the prosthetic implant is a docking device.

Example 145. The method of any example herein, particularly any one of examples 142-144, further comprising: when the retention member is coupled to the prosthetic implant: advancing a prosthetic heart valve coupled to a prosthetic heart valve delivery apparatus through the main lumen of the guide catheter to the implant location; radially expanding the prosthetic heart valve within the prosthetic implant; and removing the prosthetic heart valve from the prosthetic heart valve delivery apparatus.

Example 146. The method of any example herein, particularly any one of examples 142-145, further comprising removing the retention member from the prosthetic implant.

Example 147. The method of any example herein, particularly any one of examples 142-147, wherein advancing the retention member comprises advancing the retention member from an insertion configuration to a functional configuration, wherein the insertion configuration is one of: an external configuration, a fully internal configuration, and a partially internal configuration.

Example 148. A method for implanting a prosthetic medical device, comprising: inserting a shaft of a guide catheter into a vessel of a patient; translating a snare assembly at least partially disposed within a minor lumen of the shaft from a first position to a second position having a snare of the snare assembly distal to a distal end portion of the shaft; translating the snare assembly from the second position to a third position having the snare disposed around an outer surface of the distal end portion of the shaft; advancing a prosthetic implant coupled to a delivery apparatus through the snare and through a main lumen of the shaft to an implant location when the snare assembly is in the third position; and coupling the snare to the prosthetic implant.

Example 149. The method of any example herein, particularly example 148, further comprising removing the prosthetic implant from the delivery apparatus when the snare is coupled to the prosthetic implant.

Example 150. The method of any example herein, particularly either example 148 or example 149, wherein the prosthetic implant is a docking device.

Example 151. The method of any example herein, particularly any one of examples 148-150, further comprising: when the snare is coupled to the prosthetic implant: advancing a prosthetic heart valve coupled to a prosthetic heart valve delivery apparatus through the main lumen of the guide catheter to the implant location; radially expanding the prosthetic heart valve within the prosthetic implant; and removing the prosthetic heart valve from the prosthetic heart valve delivery apparatus.

Example 152. The method of any example herein, particularly any one of examples 148-151, further comprising removing the snare from the prosthetic implant.

Example 153. A method for implanting a prosthetic medical device, comprising: inserting a shaft of a guide catheter having a snare of a snare assembly disposed around an outer surface of the shaft into a vessel of a patient, the snare assembly partially disposed within a minor lumen of the shaft; advancing a prosthetic implant coupled to a delivery apparatus through the snare and through a main lumen of the shaft to an implant location; and coupling the snare to the prosthetic implant.

Example 154. The method of any example herein, particularly example 153, further comprising removing the prosthetic implant from the delivery apparatus when the snare is coupled to the prosthetic implant.

Example 155. The method of any example herein, particularly either example 153 or example 144, wherein the prosthetic implant is a docking device.

Example 156. The method of any example herein, particularly any one of examples 153-155, further comprising: when the snare is coupled to the prosthetic implant: advancing a prosthetic heart valve coupled to a prosthetic heart valve delivery apparatus through the main lumen of the guide catheter to the implant location; radially expanding the prosthetic heart valve within the prosthetic implant; and removing the prosthetic heart valve from the prosthetic heart valve delivery apparatus.

Example 157. The method of any example herein, particularly any one of examples 153-156, further comprising removing the snare from the prosthetic implant.

Example 158. A method for implanting a prosthetic medical device on a simulation, comprising: inserting a guide catheter into a simulation; advancing a prosthetic implant coupled to a delivery apparatus through a main lumen of the guide catheter to a target location; advancing a retention member through a minor lumen of the guide catheter; and coupling the retention member to the prosthetic implant.

Example 159. The delivery apparatus of any example herein, particularly any one of examples 1-115, 127-137, and 141, wherein the delivery apparatus is sterilized.

Example 160. The delivery assembly of any example herein, particularly any one of examples 116-126, wherein the delivery assembly is sterilized.

Example 161. The shaft of any example herein, particularly any one of examples 138-140, wherein the shaft is sterilized.

Example 162. The method of any example herein, particularly any one of examples 142-158, further comprising sterilizing the devices.

The features described herein with regard to any example can be combined with other features described in any one or more of the other examples, unless otherwise stated. For example, any one or more of the features of one shaft can be combined with any one or more features of another shaft. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A delivery apparatus comprising:
a handle;
a shaft extending distally from the handle and having a longitudinal axis, a distal end, and a proximal end, the shaft comprising a first inner surface defining a main lumen and a second inner surface defining a minor lumen; and
a retention member partially disposed within the minor lumen, the retention member comprising a retention element and an actuation element, wherein the retention member is selectively axially movable relative to the minor lumen and configured to releasably couple to a prosthetic implant at a target location.

2. The delivery apparatus of claim 1, wherein the retention element comprises one of: a snare, a loop, a lasso, claws, talons, teeth, barbs, and prongs.

3. The delivery apparatus of claim 1, wherein the actuation element comprises one of: an outer shaft, a pull shaft, and a pull wire.

4. The delivery apparatus of claim 1, wherein the shaft further comprises at least one third inner surface defining at least one pull wire lumen, wherein a pull wire is disposed within each pull wire lumen.

5. The delivery apparatus of claim 4, wherein the main lumen is coaxial with the longitudinal axis, wherein the pull wire lumen is a first radial distance from the longitudinal axis, wherein the minor lumen is a second radial distance from the longitudinal axis, wherein the second radial distance is greater than the first radial distance.

6. The delivery apparatus of claim 4, wherein the shaft comprises two third inner surfaces defining two pull wire lumens, wherein a center of the minor lumen is circumferentially between the two pull wire lumens.

7. The delivery apparatus of claim 1, wherein the shaft further defines a minor lumen exit coupled to the minor lumen, wherein the minor lumen exit comprises a distal surface.

8. The delivery apparatus of claim 7, wherein the main lumen extends axially from the distal end to the proximal end of the shaft, and wherein the minor lumen extends proximally from the minor lumen exit.

9. The delivery apparatus of claim 7, wherein the distal surface is offset axially from the distal end of the shaft by a distance D.

10. The delivery apparatus of claim 9, wherein the distance D is less than 10 centimeters.

11. The delivery apparatus of claim 7, wherein the distal surface is angled relative to the longitudinal axis.

12. The delivery apparatus of claim 7, further comprising a locking mechanism coupled to a proximal portion of the minor lumen, the locking mechanism configured to selectively lock movement of the retention member relative to the minor lumen.

13. The delivery apparatus of claim 12, wherein the locking mechanism comprises a tuohy borst adapter.

14. The delivery apparatus of claim 7, wherein the shaft further comprises a positioning structure distal to the minor lumen exit, wherein the positioning structure comprises a notch.

15. The delivery apparatus of claim 14, wherein the retention element is at least partially disposed within the notch.

16. A delivery assembly comprising:
an implant catheter including a prosthetic implant; and
a guide catheter comprising:
a shaft having a distal end and a proximal end, the shaft including a main lumen configured to receive a portion of the implant catheter therethrough and a minor lumen radially offset from the main lumen; and
a retention member at least partially disposed within the minor lumen, the retention member configured to releasably couple to the prosthetic implant.

17. A method for implanting a prosthetic medical device, comprising:
inserting a guide catheter into a vessel of a patient;
advancing a prosthetic implant coupled to a delivery apparatus through a main lumen of the guide catheter to an implant location;
advancing a retention member through a minor lumen of the guide catheter; and
coupling the retention member to the prosthetic implant.
